(19) United States
(12) Patent Application Publication
(10) Pub. No.: US 2010/0040572 A1
(43) Pub. Date: Feb. 18, 2010

(54) BLOCK ETHYLENIC COPOLYMERS COMPRISING A VINYLLACTAM BLOCK, COSMETIC COMPOSITIONS CONTAINING THEM AND COSMETIC USE OF THESE COPOLYMERS

(75) Inventor: Nathalie Mougin, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'Oreal, S.A., Paris (FR)

(21) Appl. No.: 12/428,770

(22) Filed: Apr. 23, 2009

Related U.S. Application Data
(63) Continuation of application No. 10/930,749, filed on Sep. 1, 2004, now abandoned.
(60) Provisional application No. 60/511,082, filed on Oct. 15, 2003.

(30) Foreign Application Priority Data
Sep. 1, 2003 (FR) 03 05485

(51) Int. Cl. A61K 8/81; C08L 39/04; A61Q 5/00

(52) U.S. Cl. 424/70.16; 525/205; 424/78.22

(57) ABSTRACT

Linear block ethylenic copolymer comprising:
at least one block A derived from monomers comprising from 52 to 100 % by weight of an ethylenic monomer containing a lactam ring corresponding to formula (I) below:

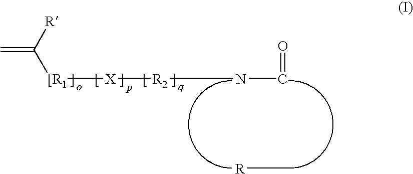

and at least one block B derived from monomers not comprising an ethylenic monomer containing a lactam ring of formula (I), or comprising a minor proportion thereof, the at least one block B having a glass transition temperature Tg ranging from −55° C. to +55° C. Compositions and processes using the compound are also disclosed.

This application claims benefit of U.S. Provisional Application No. 60/511,082, filed Oct. 15, 2003, which is herein incorporated by reference.

The present disclosure relates to novel polymers of specific structure of block ethylenic copolymer type comprising a vinyllactam block.

The present disclosure also relates to a composition, for instance a cosmetic or pharmaceutical composition, such as a hair composition, comprising the above-mentioned polymer of specific structure.

The present disclosure also relates to the cosmetic use of these polymers for treating keratin materials, such as the skin, the nails or the hair.

As used herein, the term “vinyllactam block” means a block comprising a lactam unit, for example lactam derivatives, in other words, in general, a block that can be prepared by polymerization of a monomer containing a lactam ring.

Many cosmetic compositions, such as hair compositions, referred to as “hair styling” compositions, which are in the form of aerosols (“sprays”), gels, mousses or shampoos, contain resins or polymers.

These are, for example, acrylic polymers with high glass transition temperatures (Tg), such as those described in document FR-A-2 439 798.

Such polymers may provide, especially in terms of styling, hold of the head of hair, but they may have the drawback of being excessively brittle, which does not allow good hold of the head of hair over time.

In the case of varnishes, the existing polymers may not be resistant to impacts.

To solve the problems that may be posed by these polymers, plasticizers are also used in cosmetic compositions in order to try to reduce the glass transition temperature. However, when plasticizers are used, the polymers may have a tendency to exhibit “tacky” effects or, in the case of styling, may not allow “ultra-strong” fixing.

In addition, when the hair is styled, some known hairstyling polymers may form white particles, which is unacceptable, in particular when the hair is brown and/or thick. Other drawbacks presented by the polymers currently used include possible incompatibility with known aerosol propellants.

Polymers based on vinyllactams, for example on vinylpyrrolidone, and further, for example, on homopolyvinylpyrrolidone, are moreover also known in the cosmetics field.

The main defect of these polymers is their potentially high hygroscopicity, which may lead to their having a highly tacky nature in the presence of ambient moisture.

Thus, document EP-A-1 002 811 from BASF describes water-soluble or water-dispersible grafted polymers obtained by free-radical polymerization of essentially acrylic monomers, and of a polymerizable prepolymer based on vinyllactam, for example on vinylpyrrolidone or vinylcaprolactam.

These polymers are especially used in hair compositions.

Document U.S. Pat. No. 6,193,961 from ISP describes a homogeneous terpolymer of N-vinyllactam, preferably of N-vinylpyrrolidone or N-vinylcaprolactam, of a dimethyl-aminoalkyl acrylate or of dimethylaminoalkylacrylamide and of a polysiloxane monomer.

These terpolymers are used in cosmetic and care compositions, for example cosmetic compositions such as styling gels or mousses.

This document cites numerous other patents mentioning the use of vinyllactam-based polymers in skincare and haircare compositions, such as documents U.S. Pat. No. 3,914,403, U.S. Pat. No. 3,954,960, U.S. Pat. No. 4,039,734, U.S. Pat. No. 4,057,533, U.S. Pat. No. 4,210,161, U.S. Pat. No. 4,223,009, U.S. Pat. No. 4,586,518, U.S. Pat. No. 4,764,363, U.S. Pat. No. 4,834,968, U.S. Pat. No. 4,842,850, U.S. Pat. No. 4,902,499, U.S. Pat. No. 4,906,459, U.S. Pat. No. 4,923,694, U.S. Pat. No. 4,963,348, U.S. Pat. No. 5,011,895, U.S. Pat. No. 5,015,708, U.S. Pat. No. 5,126,124, U.S. Pat. No. 5,158,762, U.S. Pat. No. 5,275,809, U.S. Pat. No. 5,502,136, WO-A-91/15186, WO-A-91/15185, EP-A2-412 704, EP-A1-0 412 707 and JP-A-57 126 409.

In this same document, it is also indicated that many patents describe the use, for instance, of an N-vinyllactam in the field of cosmetics and pharmaceutical products, e.g., in hair aerosols. These patents are documents U.S. Pat. No. 3,910,862, U.S. Pat. No. 4,923,94, U.S. Pat. No. 5,045,617, U.S. Pat. No. 5,321,110, U.S. Pat. No. 5,492,988 and U.S. Pat. No. 5,637,296.

Thus, document U.S. Pat. No. 3,954,960 relates to cosmetic and hair compositions containing, as film-forming resin, a quaternized copolymer based on vinylpyrrolidone and a copolymerizable vinyl monomer, i.e. a dialkylaminoalkyl (meth)acrylate.

As regards U.S. Pat. No. 3,914,403, it relates to hair compositions containing a mixture of film-forming resins: an N-vinylpyrrolidone homopolymer or copolymer mixed with a quaternized copolymer based on vinylpyrrolidone and a vinyl monomer that is copolymerizable therewith, i.e. a dialkylaminoalkyl (meth)acrylate.

Document U.S. Pat. No. 5,502,136 relates to a process for preparing copolymers of vinylpyrrolidone and of vinyl acetate via free-radical polymerization.

Document WO-A-00/68282 relates to terpolymers based on vinylpyrrolidone (VP), dimethylaminopropylmethacrylamide (DMAPMA), and of the DMAPMA derivative quaternized with a C₈ to C₂₄ alkyl chain, and to hair and cosmetic compositions comprising them.

It is noted that, in these copolymers, fatty chains are inserted in order to reduce the tack and to increase the moisture resistance, but the range of properties of the polymers obtained may be thus limited. Moreover, no value for the tack of these copolymers is mentioned in the above-listed documents.

The document by Matyjasezwski et al., Prepr. 38(1), 1997, p. 695, describes copolymers containing an N-vinylpyrrolidone skeleton and PDMS grafts for hydrogels.

Patent WO 97/18247 from the same K. Matyjaszewski describes, on page 103, an example of a vinylpyrrolidone homopolymer.

Document FR-A-2 327 761 relates to cosmetic compositions comprising a polymer resulting from the polymerization, in the presence of cerium, of an unsaturated monomer onto a polyvinylpyrrolidone diol. A PVP-poly(lauryl methacrylate) polymer is mentioned. The use of a vinylpyrrolidone/glucosamine acrylate copolymer is also described. The polymers of the said document are very specific polymers, generally of branched structure.

These polymers give the hairstyle sheen and hold over time.

However, they may also give the hairstyle a certain amount of rigidity, causing an unnatural appearance. They may also have the drawback of being highly hygroscopic on account of the presence of unreacted hydroxyl units along the polymer chains.

There is thus a need for a polymer, which, when included in a composition, such as a cosmetic composition, is such that this composition does not have one or more of the drawbacks, defects, limitations and disadvantages of the compositions of the prior art.

There is, in particular, a need for a polymer and a composition containing it that has an optimum combination of rigidity and tack properties.

Ideally, a hair composition comprising such a polymer would make it possible to obtain more hold, while at the same time maintaining a natural look. The polymer would, for example, in such compositions, show good styling properties and, during disentangling, would not become powdery, i.e., would not form visible “flaking” residues. Moreover, the polymer would be compatible with aerosol propellent gases.

In the case of varnishing the nails, the production of a glossy film is typically desired, this film furthermore also desirably being resistant to mechanical attack. The polymer contained in the formula would thus ideally be capable of showing excellent resistance to mechanical abrasion.

In the case of a skin treatment, the makeup used, which includes the polymer, would adhere to the skin, while at the same time being comfortable (not causing any “tautness”).

In all the cases and irrespective of the composition in which the polymer is used, it is desirable for this polymer to give a product with a non-tacky feel, in particular under conditions of high humidity.

In other words, there is a need for a film-forming polymer that has optimum rigidity properties, so as to provide access to products such as lacquers or nail varnishes with a natural effect, which have little or no “tack”.

The aim of the present disclosure is to provide a polymer that satisfies, inter alia, at least one of the needs, criteria and requirements mentioned above and which solves at least one of the problems of the polymers of the prior art.

This aim and others may be achieved, in accordance with the present disclosure, by a linear block ethylenic copolymer comprising:
at least one block A that may be derived (is obtainable) from monomers comprising from 52 to 100% by weight of an ethylenic monomer containing a lactam ring, corresponding to formula (I) below:

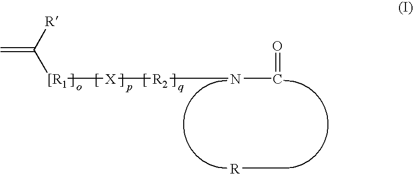

in which:
   R is a group —(CH₂)ₙ—, in which at least one of the carbon atoms is optionally replaced with a nitrogen atom or an oxygen atom, in which n is an integer from 3 to 12, and in which at least one of the carbon atoms is optionally substituted with at least one C₁ to C₆ alkyl group;
   R′ is H or a methyl group;
   R₁ and R₂, which may be identical or different, are chosen from linear, branched or cyclic alkylene or aralkylene groups of 1 to 22 C, in which at least one of the carbon atoms is optionally replaced with an oxygen or nitrogen atom;
   X is chosen from —OCO—, —NHCO—, —COO— and —O—;
   o, p and q represent, independently of each other, 0 or 1; said block A representing from 1 to 99% by weight of the copolymer;
and at least one block B that may be derived (is obtainable) from monomers not comprising an ethylenic monomer containing a lactam ring of formula (I), or comprising a minor proportion thereof, the said block B having a glass transition temperature Tg ranging from −55° C. to +55° C.

The amount of ethylenic monomer containing a lactam ring is given relative to the total weight of the monomers from which the block A may be prepared, or is obtainable.

In other words, the amount in units prepared from an ethylenic monomer containing a lactam ring of block A is from 52 to 100% by weight. Said amount of 52 to 100% by weight is given relative to the total weight of block A.

In one embodiment, in formula (I), o=0, p=1, q=1, R₂ represents —CH₂CH₂—, X represents COO or CONH and R is (CH₂)₃ or (CH₂)₅ or (CH₂CH₂NH).

The monomer of formula (I) may, for example, be a pyrrilidinoethyl methacrylate or acrylate, or a ureidoethyl acrylate or methacrylate.

In one embodiment, the block A may be prepared from monomers comprising from 52 to 100% by weight of a vinyllactam corresponding to formula (II) below:

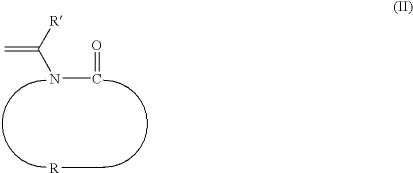

in which R and R′ have the meaning already given above.

In one embodiment, in formulae (I) and (II) above, R is —(CH₂)ₙ— with n=3 to 5, or alternatively R is —CH₂—CH₂—NH—.

A subject of the present disclosure is also cosmetic compositions comprising the said linear block ethylenic copolymers.

When they are incorporated into such compositions, the copolymers having the specific structure according to the present disclosure make it possible to obtain highly advantageous properties, or rather a combination of highly advantageous properties, which it was not possible to obtain with the polymers of the prior art.

In general, the copolymers according to the present disclosure have, on account of their particular structure, reduced hygroscopicity when compared with the copolymers of the prior art containing vinyllactam units.

The copolymers of the present disclosure have an optimum combination of rigidity and of non-tacky nature and they thus may lead to compositions or systems especially having enhanced mechanical strength, wear strength and hold over time, and reduced fragility, while at the same time not being tacky.

The copolymers according to the present disclosure may be defined as film-forming polymers, which are tack-free.

Thus, when the copolymers according to the present disclosure are used in compositions for treating the hair, such as lacquers, they may give greater hold over time. They may be less fragile than a standard lacquer and at the same time not tacky. The phenomenon of powdering on the hair, which is observed during disentangling with the compositions of the prior art, may thus be avoided.

In the case of nail varnishes, the formula comprising the copolymer according to the present disclosure has greater wear strength and is not tacky, while at the same time adheres to the nail. As a result, the loss of gloss, i.e. the matting of the film by mechanical marking or by marking with dust which takes place with tacky films of the prior art, does not take place with the varnishes and films comprising the copolymer according to the present disclosure. The reason for this is that the absence of tack from these copolymers has the effect that they are not mechanically marked, they do not retain dust and they therefore do not undergo changes in or loss of gloss.

The varnishes comprising the copolymer according to the present disclosure give films that do not chip, especially by virtue of the presence in the copolymer according to the present disclosure of a block B having a very particular Tg, within a precise range, which prevents the films from chipping, but at the same time, and surprisingly, does not lead to a tacky film.

In makeup products, for instance lipsticks or foundations, the makeup shows good staying power on the lips or the skin, without giving a tacky sensation.

The present disclosure also relates to a cosmetic process for making up or caring for keratin materials, comprising the application to the keratin materials of a composition according to the present disclosure.

The present disclosure thus also relates to the use of the copolymers according to the present disclosure to improve the hairstyle hold, without tack, of a hair lacquer; the use of the copolymers according to the present disclosure to improve the adhesion and the wear resistance, without tack, of a nail-varnish; and, finally, the use of the copolymers according to the present disclosure in a cosmetic composition such as a makeup composition for masking wrinkles, which gives the skin a smoothened appearance, without tautness.

The copolymers according to the present disclosure thus provide a solution to at least some of the problems posed by the polymers of the prior art.

The unexpected advantageous properties of the specific copolymers of the present disclosure, which are fundamentally linear copolymers, may arise especially, or even essentially, from the specific nature of the blocks of which they are made.

Surprisingly, by virtue, so it would appear, of the presence of a block (block B) free of ethylenic monomer containing a lactam ring such as a vinyllactam, or in which the ethylenic monomer containing a lactam ring such as a vinyllactam is in minor amount, the copolymers according to the present disclosure are less hygroscopic than the vinyllactam-based polymers of the prior art and especially than vinyllactam homopolymers, such as homopolyvinylpyrrolidone.

In addition, it has been demonstrated, surprisingly, according to the present disclosure, that the presence in the polymer according to the present disclosure of specific blocks B with very specific Tg values makes it possible to control the brittleness of the copolymer and obtain, surprisingly, copolymers that are not fragile or brittle, which give flexible films and which do not powder, while at the same time not being tacky; one of the essential drawbacks of the polyvinyllactam-based polymers of the prior art that are generally relatively brittle, while at the same time being tacky, may thus be overcome.

There was nothing in the prior art to suggest that by using a specifically linear copolymer, stipulating that at least one block (A) of this copolymer should comprise from 52 to 100% by weight of an ethylenic monomer containing a lactam ring such as a vinyllactam, that at least one other block (B) should have a structure different from the block (A) and should be free of or consist in minor amount of an ethylenic monomer containing a lactam ring such as an N-vinyllactam, and finally that the block B should have a specific glass transition temperature ranging from −55° C. to +55° C., it would be possible, according to the present disclosure, to obtain a combination of excellent properties for this copolymer. It is believe that the specific structure of the copolymer of the present disclosure leads to optimization of its properties, leading to equilibrium between the mechanical strength and non-tack properties.

Without wishing to be bound by any theory, the advantageous properties of the copolymer according to the present disclosure are thought to arise from the fact that the nature of the blocks is specifically chosen so as to promote the separation of the phases from each other and thus, inter alia, to give optimum control of the rigidity and the hygroscopicity and thus of the tack of the copolymer. Moreover, the fact that the polymer is linear entails a much simpler and much more controlled synthesis, which makes it possible to precisely predict the structure of the polymers obtained, and thus to optimize the final properties of the polymers.

More specifically, the copolymers according to the present disclosure are block copolymers. As used herein, this term means that the copolymers comprise blocks that are covalently attached to each other.

In addition, two successive blocks may be of different nature. In contrast, two non-successive blocks A or B may be of the same nature. Each block A or B may comprise a homopolymer or a copolymer, it being possible for the latter in turn to be a random copolymer, an alternating copolymer or a gradient copolymer.

The polymer may also comprise another block C other than the blocks A and B and optionally yet other blocks, for example a block D other than A, B and C.

The polymer disclosed herein may thus be chosen from diblock copolymers of AB type or triblock copolymers of ABA, BAB, ABC or ACB type, with C other than A or B.

The polymer disclosed herein may also be chosen from multiblock copolymers containing more than three identical or different blocks: (AB)ₙ, (ABA)ₙ, (BAB)ₙ, (ABC)ₙ or (ABC)ₙ, with C other than A or B, or multiblock copolymers containing more than three different blocks ABCD.

In general, as has already been mentioned, the nature of the blocks may be chosen so as to promote the phase separation between the blocks, since this may predetermine the properties.

The nature and number of the blocks are chosen by a person skilled in the art as a function of the desired properties within the limits of the conditions specified above for blocks A and B.

The copolymers of the present disclosure are defined as being ethylenic copolymers. This means that the monomers from which the blocks constituting the copolymer are derived are monomers containing a carbon-carbon unsaturated double bond of ethylenic type.

In addition, the copolymer disclosed herein is a linear copolymer. This means that the present disclosure is not intended to cover copolymers having a non-linear structure, for example a branched, starburst, grafted or other structure. The linear nature of the copolymers disclosed herein is what is believed to give the compositions containing it the advantageous properties described above.

In one embodiment, the copolymer is a film-forming polymer, i.e., it is capable by itself, or in the presence of an auxiliary film-forming agent, at a temperature ranging from 20° C. to 30° C., of forming a continuous film (viewed by the naked eye) and adhering to a keratin support.

According to the present disclosure, the copolymer comprises at least one block A that may be prepared or derived from monomers comprising from 52 to 100% by weight of an ethylenic monomer containing a lactam ring, for example a vinyllactam, of formula (I) and for example of formula (II).

The amount of ethylenic monomer containing a lactam ring is given relative to the total weight of the monomers from which the block A may be prepared, i.e., is obtainable.

In other words, the amount in units prepared from an ethylenic monomer containing a lactam ring of block A is from 52 to 100% by weight. Said amount of 52 to 100% by weight is given relative to the total weight of block A.

In general, the percentage of ethylenic monomer containing a lactam ring, such as a vinyllactam, of formula (I) or (II) in the monomers from which the block A may be prepared (is obtainable), may range from 55% to 95% by weight, for example it is 90% by weight.

In other words, the amount in units prepared from an ethylenic monomer containing a lactam ring may range from 55 to 95% by weight, for example 90% by weight, these weight contents are given relative to the total weight of the block A.

The block A may have any glass transition temperature Tg, but it generally has an overall glass transition temperature of the “high” block. The term “high” generally means that this Tg may range from 0 to 250° C., such as from 0 to 220° C. and for example, from 5 to 200° C.

For example, the glass transition temperature Tg of a block A comprising homopolyvinyllactam is generally very much greater than 100° C. at an RH value of 0%.

The block A may be hydrophilic or hydrophobic. A hydrophilic block may be defined as being a water-soluble or water-dispersible block. In one embodiment, the block A is a hydrophilic block. The term “hydrophilic” is defined below.

In one embodiment, the block A is a homopolymer of the ethylenic monomer containing a lactam ring, such as N-vinyllactam, of formula (I) or of formula (II).

The N-vinyllactam monomer may be chosen from N-vinyllactams and derivatives thereof, which may contain, for example, at least one at least one C₁ to C₆ alkyl substituent, such as methyl, ethyl, n-propyl, isopropyl, n-butyl or sec-butyl.

For example, the said N-vinyllactam of formula (I) may be pyrrilidinoethyl acrylate or pyrrilidinoethyl methacrylate.

For example, the N-vinyllactam of formula (II) is N-vinylpyrrolidone (n=3), N-vinylpiperidinone (valerolactam) (n=4), N-vinylcaprolactam (n=5), N-vinylimidazolidinone in which R is a —CH₂—CH₂—NH— group, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam or N-vinyl-7-ethyl-2-caprolactam.

In one embodiment, with respect to the choice of the N-vinyllactam, when the block A is a homopolymer, it is a homopolymer of N-vinylpyrrolidone or of N-vinylcaprolactam.

When the block A, is a copolymer, the monomers from which it may be prepared may comprise, besides the ethylenic monomer containing a lactam ring, such as the vinyllactam of formula (I), at least one at least one other monomer.

For example, independently of the choice of the N-vinyllactam, the block A is a copolymer since it makes it possible to modify the properties and in particular to modify the tack properties.

This or these monomers of the block A, other than the ethylenic monomer containing a lactam ring, such as vinyllactam, are ethylenic monomers that are copolymerizable with the lactam derivative monomer, irrespective of the coefficient of reactivity thereof.

This or these monomer(s) of the block A, other than the ethylenic monomer containing a lactam ring, such as vinyllactam, are, for example, chosen from monomers for which the Tg of the corresponding homopolymer is less than or equal to 50° C., such as less than or equal to 20° C. and further such as less than or equal to 0° C.; in one embodiment, the monomers also have a Tg for the corresponding homopolymer that is greater than or equal to −150° C.

In the block A, the monomer(s) other than the ethylenic monomer containing a lactam ring, for instance a vinyllactam, may be hydrophilic or non-hydrophilic.

For instance, in the block A, at least one from among the monomers other than the ethylenic monomer containing a lactam ring, for instance a vinyllactam, is chosen from hydrophilic monomers.

The hydrophilic monomers are chosen, for example, from the hydrophilic monomers described below. Examples of hydrophilic monomers include the cationic monomers, anionic monomers and nonionic monomers described below.

At least one monomers other than the ethylenic monomer containing a lactam ring, for instance a vinyllactam, may be chosen from non-hydrophilic monomers. These non-hydrophilic monomers are chosen from the list of monomers that can constitute the block B, while respecting the desired Tg and hydrophilicity criteria.

In one embodiment, monomer(s) other than the ethylenic monomer containing a lactam ring, for instance a vinyllactam, for which the Tg of the corresponding homopolymer is less than or equal to 50° C., is(are) chosen from:

t-butyl acrylate (Tg=50° C.), vinyl acetate (Tg=23° C.), butyl methacrylate (Tg=20° C.), cyclohexyl acrylate (Tg=19° C.), hydroxyethyl acrylate (Tg=15° C.), methyl acrylate (Tg=10° C.), ethoxyethyl methacrylate (Tg=0° C.), n-hexyl methacrylate (Tg=−5° C.), ethyl acrylate (Tg=−24° C.), isobutyl acrylate (Tg=−24° C.), methyl vinyl ether (Tg=−34° C.), methoxyethyl acrylate (Tg=−33° C.), n-butyl acrylate (Tg=−54° C.), ethylhexyl acrylate (Tg=−50° C.), POE methacrylate (n=8 to 10) (Tg=−55° C.) and isobornyl methacrylate; butylacrylamide, N-isopropylacrylamide, N,N-dimethylacrylamide and N,N-dibutylacrylamide, hexyl acrylate, octyl acrylate, lauryl acrylate, isooctyl acrylate, ethylhexyl methacrylate, octyl methacrylate, lauryl methacrylate, vinyl neononanoate and vinyl neododecanoate; N-octylacrylamide.

The block A, comprising from 52 to 100% by weight of ethylenic monomer containing a lactam ring, represents from 1% to 99% by weight of the copolymer, such as from 10% to 95% and further such as from 20% to 90% by weight relative to the total weight of the final copolymer.

The block(s) (B) other than the block A may be prepared or derived from at least one ethylene monomer generally chosen from: allylic monomers, acrylates, methacrylates, acrylamides, methacrylamides and vinyl monomers, and mixtures thereof, and optionally ethylenic monomers containing a lactam ring, for example vinyllactam monomers, of formula (I) or (II), the weight proportion of the said monomers (I) or (II) being a minor proportion, i.e. less than 50% by weight, such as less than or equal to 40% and further such as less than or equal to 30% by weight.

In one embodiment, the number-average molecular mass of each block, whether it is block A or block B, ranges from 2000 to 1 000 000, such as from 2000 to 800 000 and further such as from 2000 to 500 000.

For instance, the copolymer may comprise at least one block that has a specific glass transition temperature in the range from −55° C. to +55° C.

Since the glass transition temperature Tg is a parameter used herein for defining the blocks of the copolymer of the present disclosure, especially block B of the copolymer of the present disclosure and, consequently, the copolymer of the present disclosure, it is important to point out that the glass transition temperatures of the blocks of the copolymers used in the present disclosure are measured by differential thermal analysis (DSC, “Differential Scanning Calorimetry”) for the dry polymer, at a heating rate of 10° C./minute.

Each block of the copolymer according to the present disclosure is derived from one type of monomer or from several different types of monomer.

This means that each block may consist of a homopolymer or a copolymer; this copolymer constituting the block may in turn be a random or alternating or gradient copolymer. In the case of block A it is a vinyllactam homopolymer or a copolymer comprising a proportion of at least 52% by weight of ethylenic monomer containing a lactam ring, such as a vinyllactam.

The number-average molecular mass of the overall copolymer (for example A-b-B) generally ranges from 4000 to 1 000 000, such as from 4000 to 800 000 and further such as from 4000 to 500 000.

In one embodiment, the proportion of the block B with an overall Tg of from −55° C. to +55° C. is from 1% to 99% by weight of the copolymer, such as from 5% to 90% and further such as from 10% to 80% of the total weight of the final polymer.

In one embodiment, the block B whose Tg value may range from −55° C. to 55° C. has a Tg temperature that can vary within the range from −50° C. to +50° C. such as from −50° C. to +45° C.

For example, the monomer(s) constituting the block B that satisfies the above Tg condition may have a carbon chain length of less than or equal to 10 carbon atoms; the reason for this is that this will have a tendency to reduce the tack of the block under consideration, or alternatively, if the carbon chains have a carbon chain length of greater than or equal to 12 carbon atoms, the proportion of these monomers in the block B is low, i.e. generally less than 50% by weight. The term “carbon chain” as used herein means the chain borne by the ethylenic unit and thus pendent on the skeleton.

The block B, the overall glass transition temperature of which (presented by the block taken as a whole) ranges from −55° C. to +55° C., is a homopolymer or copolymer, derived from at least one monomer which is such that the homopolymers prepared from these monomers may have glass transition temperatures ranging from −200 to +250° C.

A person skilled in the art chooses these monomers and the proportions thereof such that the overall Tg of the block is within the desired range.

A block B with a Tg of from −55 to +55° C. may thus be formed from a copolymer consisting of a first monomer for which the Tg of the corresponding homopolymer is in the range from +20° C. to +250° C., such as methyl methacrylate (MMA) or styrene, and of a second monomer for which the Tg of the corresponding homopolymer is in the range from −200° C. to +20° C.

For example, it will be possible to combine in the copolymer, forming the block, a monomer with a Tg (for the corresponding homopolymer) equal to 100° C., for example methyl methacrylate, in a proportion of 35% by weight relative to the total weight of monomers, and a monomer with a Tg equal to −50° C., for example 2-ethylhexyl acrylate, in a proportion of 65% by weight, and the resulting block will have a Tg of −14° C., calculated to a first approximation by Fox's law: 1/Tg=sum (% Wa/Tga) with Wa=% by weight of the monomer a and Tga=glass transition temperature of the homopolymer made from the monomer a.

The block B whose glass transition temperature may range from −55° C. to +55° C. may also be a homopolymer, made from only one type of monomer for which the Tg of the corresponding homopolymer is within the range from −55° C. to +55° C., such as from 50 to +50° C. and further such as from −50 to +45° C. It should be noted, however, that there are few monomers for which the Tg of the corresponding homopolymer is in this range: most of them are mentioned below.

The monomer(s) from which the block(s) B may be prepared may thus be chosen, for example, from the monomers described below.
ethylenic hydrocarbons of 2 to 10 C, such as ethylene, isoprene or butadiene;
the acrylates of formula CH₂═CHCOOR₃;
the methacrylates of formula:

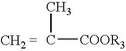

in which R₃ represents:
   a linear or branched alkyl group of 1 to 18 carbon atoms in which at least one hetero atom chosen from O, N, S and P is optionally intercalated, the said alkyl group also possibly being optionally substituted with at least one substituent chosen from hydroxyl groups, halogen atoms (Cl, Br, I and F) and groups Si(R₄R₅), in which R₄ and R₅, which may be identical or different, are chosen from a C₁ to C₆ alkyl group and a phenyl group;
      examples of these alkyl groups are methyl, ethyl, propyl, butyl, isobutyl, tert-butyl, ethylhexyl, octyl, lauryl and stearyl,
      examples of these alkyl-based groups (i.e. substituted and/or interrupted alkyl) are C₁₋₄ hydroxyalkyl groups such as 2-hydroxyethyl and 2-hydroxypropyl, and (C₁₋₄)alkoxy (C₁₋₄) alkyl groups such as methoxyethyl, ethoxyethyl and methoxypropyl,
a C₃ to C₁₂ cycloalkyl group, such as an isobornyl group,
a C₃ to C₂₀ aryl group such as a phenyl group,
a C₄ to C₃₀ aralkyl group (C₁ to C₈ alkyl group) such as 2-phenylethyl or benzyl,
a 4- to 12-membered heterocyclic group containing at least one hetero atom chosen from O, N and S, the ring being aromatic or non-aromatic,
a heterocyclylalkyl group (1 to 4 C alkyl), such as furfurylmethyl or tetrahydrofurfuryl methyl,
   the said cycloalkyl, aryl, aralkyl, heterocyclic or heterocyclylalkyl groups possibly being optionally substituted with at least one substituent chosen from hydroxyl groups, halogen atoms and linear or branched alkyl groups of 1 to 4 C in which at least one hetero atom chosen from O, N, S and P is optionally intercalated, the said alkyl groups also possibly being optionally substituted with at least one substituent chosen from hydroxyl groups, halogen atoms (Cl, Br, I and F) and groups Si(R₄R₅), in which R₄ and R₅, which may be identical or different, are chosen from a C₁ to C₆ alkyl group and a phenyl group, examples of groups R₃ are methyl, ethyl, propyl, isobutyl, n-butyl, tert-butyl, isobutyl, hexyl, ethylhexyl, octyl, lauryl, isooctyl, isodecyl, dodecyl, cyclohexyl, t-butylcyclohexyl, t-butylbenzyl, isobornyl, phenyl, furfurylmethyl, tetrahydrofurfurylmethyl, 2-hydroxyethyl, 2-hydroxypropyl, 2-hydroxybutyl, methoxyethyl, ethoxyethyl, methoxyethyl, methoxypropyl and 2-ethylperfluorohexyl groups,
another example of a group R₃ for the acrylates is the groups R₃=—(OC₂H₄)ₘ—OR″, with m=5 to 150 and R″=H or C₁ to C₃₀ alkyl, for example —POE-methoxy and —POE-behenyl;
the (meth)acrylamides of formula:

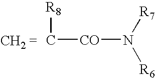

in which
R₈ is H or methyl;
and R₇ and R₆, which may be identical or different, are each chosen from a hydrogen atom and linear or branched alkyl groups of 1 to 18 carbon atoms, in which at least one hetero atom chosen from O, N, S and P is optionally intercalated, the said alkyl group also possibly being optionally substituted with at least one substituent chosen from hydroxyl groups, halogen atoms (Cl, Br, I and F) and groups Si(R₄R₅), in which R₄ and R₅, which may be identical or different, represent a C₁ to C₆ alkyl group or a phenyl group,
   examples of these groups are methyl, ethyl, n-butyl, t-butyl, isopropyl, isohexyl, isooctyl, isononyl and C₁₋₄ hydroxyalkyl groups such as 2-hydroxypropyl,
a C₃ to C₁₂ cycloalkyl group, such as an isobornyl group,
a C₃ to C₂₀ aryl group such as phenyl
a C₄ to C₃₀ aralkyl group (C₁ to C₈ alkyl group) such as 2-phenylethyl or benzyl,
a 4- to 12-membered heterocyclic group containing at least one hetero atom chosen from O, N and S, the ring being aromatic or non-aromatic,
a heterocyclylalkyl group (1 to 4 C alkyl), such as furfurylmethyl or tetrahydrofurfurylmethyl,
   the said cycloalkyl, aryl, aralkyl, heterocyclic or heterocyclylalkyl groups possibly being optionally substituted with at least one substituent chosen from hydroxyl groups, halogen atoms and linear or branched alkyl groups of 1 to 4 C in which at least one hetero atom chosen from O, N, S and P is optionally intercalated, the said alkyl groups also possibly being optionally substituted with at least one substituent chosen from hydroxyl groups, halogen atoms (Cl, Br, I and F) and groups Si(R₄R₅), in which R₄ and R₅, which may be identical or different, are chosen from a C₁ to C₆ alkyl group and a phenyl group.

Examples of (meth)acrylamide monomers are (meth)acrylamide, N-ethyl(meth)acrylamide, N-butylacrylamide, N-t-butylacrylamide, N-isopropylacrylamide, N,N-dimethyl(meth)acrylamide, N,N-dibutylacrylamide, N-octylacrylamide, N-dodecylacrylamide, undecylacrylamide and N(2-hydroxypropylmethacrylamide.
the allylic compounds of formula:

CH₂═CH—CH₂—R₉ or CH₂═C(CH₃)—CH₂—R₉;
the vinyl compounds of formula:

CH₂═CH—R₉,
in which R₉ is a group:
hydroxyl,
Cl,
NH₂,
OR₁₀, in which R₁₀ is a phenyl group or a C₁ to C₁₂ alkyl group (the monomer is a vinyl ether or an allyl ether),
Acetamide: NHCOCH₃,
OCOR₁₁, in which R₁₁ is chosen from:
   a linear or branched alkyl group of 2 to 12 C (the monomer is a vinyl ester or an allylic ester),
   a C₃ to C₁₂ cycloalkyl group such as isobornyl or cyclohexyl,
   a C₃ to C₂₀ aryl group such as phenyl,
   a C₄ to C₃₀ aralkyl group (C₁ to C₈ alkyl group) such as 2-phenylethyl or benzyl,
   a 4- to 12-membered heterocyclic group containing at least one hetero atom chosen from O, N and S, the ring being aromatic or non-aromatic,
   a heterocyclylalkyl group (1 to 4 C alkyl), such as furfurylmethyl or tetrahydrofurfurylmethyl,
      the said cycloalkyl, aryl, aralkyl, heterocyclic or heterocyclylalkyl groups possibly being optionally substituted with at least one substituent chosen from hydroxyl groups, halogen atoms and linear or branched alkyl groups of 1 to 4 C in which at least one hetero atom chosen from O, N, S and P is optionally intercalated, the said alkyl groups also possibly being optionally substituted with at least one substituent chosen from hydroxyl groups, halogen atoms (Cl, Br, I and F) and groups Si(R₄R₅), in which R₄ and R₅, which may be identical or different, are chosen from a C₁ to C₆ alkyl group and a phenyl group.

Examples of vinyl monomers are vinylcyclohexane and styrene.

Examples of vinyl esters are: vinyl acetate, vinyl propionate, vinyl butyrate, vinyl ethylhexanoate, vinyl neononanoate and vinyl neododecanoate.

Among the vinyl ethers are, for example, methyl vinyl ether, ethyl vinyl ether and isobutyl vinyl ether;
(meth)acrylic or (meth)acrylamide or vinyl monomers containing a fluoro or perfluoro group, such as ethylperfluorooctyl methacrylate;
silicone (meth)acrylic or vinyl monomers, such as methacryloxypropyltris(trimethylsiloxy)silane, acryloxypropylpolydimethylsiloxane or silicone (meth)acrylamides.

In one embodiment, monomers for the block B are those for which the glass transition temperature Tg of the corresponding homopolymer is in the range from −55° C. to +55° C., such as: hydroxyethyl methacrylate (Tg=55° C.), isobutyl methacrylate (Tg=53° C.), n-hexyl acrylate (Tg=45° C.), t-butyl acrylate (Tg=50° C.), vinyl acetate (Tg=23° C.), butyl methacrylate (Tg=20° C.), cyclohexyl acrylate (Tg=19° C.), hydroxyethyl acrylate (Tg=15° C.), methyl acrylate (Tg=10° C.), ethoxyethyl methacrylate (Tg=0° C.), n-hexyl methacrylate (Tg=−5° C.), vinyl butyrate (Tg=−5° C.), ethyl acrylate (Tg=−24° C.), isobutyl acrylate (Tg=−24° C.), methyl vinyl ether (Tg=−34° C.), methoxyethyl acrylate (Tg=−33° C.), n-hexyl acrylate (Tg=−45° C.), n-butyl acrylate (Tg=−54° C.), ethylhexyl acrylate (Tg=−50° C.) and POE methacrylate (n=8 to 10) (Tg=−55° C.).

Among the other monomers whose Tg varies outside the range from −55 to +55° C., examples that may be mentioned include the following monomers: t-butylcyclohexyl acrylate, t-butylbenzyl acrylate, furfuryl acrylate and isobornyl acrylate; methyl methacrylate, ethyl methacrylate, cyclohexyl methacrylate, t-butylcyclohexyl methacrylate, t-butylbenzyl methacrylate and isobornyl methacrylate;

N-butylacrylamide, N-t-butylacrylamide, N-isopropylacrylamide, N,N-dimethylacrylamide and N,N-dibutylacrylamide;

vinylcyclohexane, styrene;

hexyl acrylate, octyl acrylate, lauryl acrylate, isooctyl acrylate and isodecyl acrylate;

ethylhexyl methacrylate, octyl methacrylate, lauryl methacrylate, isooctyl methacrylate and isodecyl methacrylate;

vinyl neononanoate and vinyl neododecanoate;

N-octylacrylamide.

The block B with an overall Tg ranging from −55° C. to +55° C. may thus, for example, consist totally or partially of at least one monomer, mentioned above, for which the glass transition temperature of the corresponding homopolymer is in the range from −55° C. to +55° C. If the block B consists totally of such monomers, it may be a homopolymer or a copolymer which then consists of at least two monomers, such that the block B has overall only one Tg rather than, for example, two different separate Tg values: Tg₁ and Tg₂ which each correspond to a glass transition temperature of the homopolymers corresponding to each of the two monomers forming the block B.

The block B may consist only partially of at least one monomer for which the glass transition temperature of the corresponding homopolymer is in the range from −55° C. to +55° C.

In this case, the block with a glass transition temperature of from −55° C. to +55° C. may, in addition to the monomers indicated above, and which the glass transition temperature Tg of the corresponding homopolymer is in the range from −55° C. to +55° C., for example less than or equal to 20° C., comprise at least one other monomer. These other monomers are monomers for which the glass transition temperature Tg of the corresponding homopolymer is outside the range −55° C. to +55° C.

This or these monomer(s) is(are) chosen, of course, such that the overall Tg of the block is within the range indicated above.

The block B may also be a copolymer, consisting totally of monomers for which the glass transition temperature Tg of the corresponding homopolymer is outside the range from −55° C. to +55° C.

These monomers are chosen, of course, such that the block B has overall a glass transition temperature that is in the range from −55° C. to +55° C.

The monomers that are particularly preferred, for which the Tg varies in the range from −55 to 55° C., among all those mentioned above, are chosen from n-hexyl acrylate (Tg=45° C.), t-butyl acrylate (Tg=50° C.), vinyl acetate (Tg=23° C.), butyl methacrylate (Tg=20° C.), hydroxyethyl acrylate (Tg=15° C.), cyclohexyl acrylate (Tg=19° C.), methyl acrylate (Tg=10° C.), vinyl butyrate (Tg=−5° C.), methyl vinyl ether (Tg=−34° C.), n-butyl acrylate (Tg=−54° C.), ethylhexyl acrylate (Tg=−50° C.), isobutyl acrylate (Tg=−24° C.), methoxyethyl acrylate (Tg=−33° C.), n-hexyl methacrylate (Tg=−5° C.), POE methacrylate (n=8 to 10) (Tg=−55° C.) and hydroxypropyl acrylate.

In one embodiment, at least one monomer from among the monomers constituting the block(s) B is chosen from hydrophilic monomers. In one embodiment, at least one from among the blocks B other than the blocks A is hydrophilic and contains hydrophilic monomers.

In one embodiment, the copolymer according to the present disclosure comprises at least one hydrophilic block that comprises hydrophilic monomers. This or these block(s) may be the block that comprises from 52 to 100% by weight of ethylenic monomer containing a lactam ring, such as a vinyllactam, and/or at least one of the other blocks that constitute the polymer, for example at least one of the blocks B.

The hydrophilic block may be defined as being a water-soluble or water-dispersible block.

The polymer forming the block is water-soluble if it is soluble in water to a proportion of at least 5% by weight, at 25° C.

The polymer forming the block is water-dispersible if it forms, at a concentration of 5%, at 25° C., a stable suspension of generally spherical fine particles. The mean size of the particles constituting the said dispersion is less than 1 μm and more generally ranges from 5 to 400 nm, such as from 10 to 250 nm. These particle sizes are measured by light scattering.

The hydrophilic block may be a block whose glass transition temperature is high, for example from 0 to 250° C., such as from 50 to 200° C.
Examples of hydrophilic monomers include cationic monomers, anionic monomers and nonionic monomers:
Examples of cationic monomers are:
   2-vinylpyridine (Tg: 104° C.);
   4-vinylpyridine (Tg: 142° C.);
   dimethylaminoethyl (meth)acrylate (Tg: 19° C.);
   diethylaminoethyl (meth)acrylate;
   dimethylaminopropyl (meth)acrylamide; and salified or quaternized forms thereof, whether they are salts of mineral acids, such as sulfuric acid or hydrochloric acid, or salts of organic acids.

These organic acids may comprise at least one carboxylic, sulfonic or phosphonic acid group. They may be linear, branched or cyclic aliphatic acids or alternatively aromatic acids. These acids may also comprise at least one hetero atom chosen from O and N, for example in the form of hydroxyl groups.

An example of an acid containing an alkyl group is acetic acid, CH₃COOH.

An example of a polyacid is terephthalic acid.

Examples of hydroxy acids are citric acid and tartaric acid.
Examples of anionic monomers are:
   acrylic acid, methacrylic acid, crotonic acid, maleic anhydride, itaconic acid, fumaric acid and maleic acid;
   styrenesulfonic acid, 2-acrylamido-2-methylpropanesulfonic acid, vinylbenzoic acid, vinylphosphoric acid and sulfopropyl methacrylate, and the salts thereof.

The neutralizer may be a mineral base, such as LiOH, NaOH, KOH, Ca(OH)₂ or NH₄OH, or an organic base. It may be a primary, secondary or tertiary alkylamine, such as triethylamine or butylamine. This primary, secondary or tertiary alkylamine may also comprise nitrogens or oxygens and may thus comprise, for example, an alcohol function, for example, 2-amino-2-methylpropanol or triethanolamine.
Examples of nonionic monomers are:
   ethylenic carboxybetaines or sulfobetaines obtained, for example, by quaternization of ethylenically unsaturated monomers comprising an amine function with carboxylic acid salts containing a labile halogen, for example sodium chloroacetate, or with cyclic sulfones, for example propane sulfone;
   hydroxyalkyl (meth)acrylates or hydroxyalkyl(meth)acrylamides, the alkyl group of which contains from 2 to 4 C atoms, in particular hydroxyethyl (meth)acrylate or hydroxypropyl (meth)acrylate;
   (meth)acrylates or (meth)acrylamides of (C₁-C₄)alkoxy(C₁₋₄)alkyl, such as methoxyethyl, ethoxyethyl and methoxypropyl,
   (meth)acrylates or (meth)acrylamides containing a group —(OC₂H₄)ₘ—OR′″, in which m=5 to 150 and R′″=H or C₁ to C₄ alkyl, for example —POE-methoxy; —POE-OH;
   vinyllactams, and
   polysaccharide (meth)acrylates, for instance sucrose acrylate.

However, the vinyllactams can constitute only a minor proportion of the block B.

It should be noted that even though the copolymer comprises a hydrophilic block, the overall copolymer is not necessarily hydrophilic.

The linear block ethylenic copolymers according to the present disclosure are chosen from:
diblock copolymers (AB),
triblock copolymers (ABA, BAB, ABC or ACB), with C other than A or B,
multiblock copolymers containing more than three blocks: (AB)ₙ, (ABA)ₙ, (BAB), or (ABC)ₙ, with C other than A or B, or multiblock copolymers containing more than three different blocks, of ABCD type.

The copolymers according to the present disclosure may be prepared by anionic polymerization.

In one embodiment, however, the copolymers according to the present disclosure are obtained in a first mode by controlled free-radical polymerization and, in a further embodiment, the copolymers according to the present disclosure may be obtained via a particular “ATRP” polymerization, i.e. the technique known as “Reverse ATRP”, or via the “RAFT” technique, but the polymers according to the present disclosure may, according to a second mode, also be obtained via standard free-radical polymerization.

### First Mode

The block copolymers according to the present disclosure may, for example, be obtained by controlled free-radical polymerization, described especially in “New Method of Polymer Synthesis”, Blackie Academic Professional, London, 1995, Volume 2, page 1.

Controlled free-radical polymerization may make it possible to reduce the deactivation reactions of the growing free-radical species, such as the termination step, these being reactions which, in standard free-radical polymerization, may irreversibly and uncontrollably stop the growth of the polymer chain.

In order to reduce the probability of the termination reactions, it has been proposed to temporarily and reversibly block the growing free-radical species, by forming “dormant” active species in the form of a bond with a low dissociation energy.

Thus, the polymerization may be performed according to the Atom Transfer Radical Polymerization (ATRP) technique, or by reaction with a nitroxide, or alternatively according to the “reversible addition-fragmentation chain transfer” (RAFT) technique, or, finally, by the “reverse ATRP” technique.

The atom transfer radical polymerization technique, also known as ATRP, involves blocking the growing free-radical species in the form of a bond of C-halide type (in the presence of a metal/ligand complex). This type of polymerization may be reflected by control of the mass of the polymers formed and by a low dispersity index of the masses.

In general, the atom transfer radical polymerization is performed by polymerization of at least one free-radical-polymerizable monomer, in the presence of:
an initiator containing at least one transferable halogen atom;
a halogenated compound comprising a transition metal capable of participating in a reduction step with the initiator and a “dormant” polymer chain, this compound will be referred to as the “chain-transfer agent”; and
a ligand that may be chosen from compounds comprising a nitrogen (N), oxygen (O), phosphorus (P) or sulfur (S) atom, capable of coordinating via a bond to the said compound comprising a transition metal, the formation of direct bonds between the said compound comprising a transition metal and the polymer under formation being avoided.

The halogen atom is, for example, a chlorine or bromine atom.

This process is described in particular in WO 97/18247 and in the article by Matyjasezwski et al. published in JACS, 117, page 5614 (1995).

The technique of free-radical polymerization by reaction with a nitroxide involves blocking the growing free-radical species in the form of a bond of C—O—NRₐR_{b} type, in which Rₐ and R_{b} may be, independently of each other, an alkyl radical containing from 2 to 30 carbon atoms, or together forming, with the nitrogen atom, a ring containing from 4 to 20 carbon atoms, for instance a 2,2,6,6-tetramethylpiperidyl ring. This polymerization technique is described in detail in the articles “Living free radical polymerization: a unique technique for preparation of controlled macromolecular architectures”, CJ Hawker; Chem. Res., 1997, 30, 373-82 and “Macromolecular engineering via living free radical polymerizations” published in Macromol. Chem. Phys. 1998, Vol. 199, pages 923-935, or alternatively in patent application WO-A-99/03894.

The RAFT (reversible addition-fragmentation chain transfer) polymerization technique involves blocking the growing free-radical species in the form of a bond of C—S type. Dithio compounds such as dithioesters (—C(S)S—), such as dithiobenzoates, dithiocarbamates (—NC(S)S—) or dithiocarbonates (—OC(S)S—) (xanthates) are used to do this. These compounds make it possible to control the chain growth of a wide range of monomers. However, dithioesters inhibit the polymerization of vinyl esters, while dithiocarbamates are very weakly active towards methacrylates, which limits the application of these compounds to a certain extent. This technique is described for instance in patent application WO-A-98/58974 from Rhodia and in the article “A more versatile route to block copolymers and other polymers of complex architecture by living radical polymerization: the RAFT process”, published in Macromolecules, 1999, Volume 32, pages 2071-2074. The already mentioned patent application WO-A-98/58974 and the patent application WO-A-99/31144 from CSIRO relate to the use of dithiocarbamates as “RAFT” reagents. By using these dithiocarbamates, various monomers are polymerized, including vinyl acetate.

The main advantages of the “RAFT” technique over the “ATRP” technique are that it does not require a metal catalyst and also that standard radical initiators may be used to initiate the reaction.

In the case of controlled free-radical polymerization, for example of >N-vinylpyrrolidone (NVP), the chain-transfer agents which may be used in the case of “RAFT” polymerization are diphenyldithiocarbamate derivatives.

By varying the ratio of the concentration of monomer to the concentration of chain-transfer agent, the molecular mass of the polymer may be modified.

The polymerization proceeds in several steps according to the general scheme:
a—in a first step, polymerization of the first monomer or mixture of monomers is performed to form a macroinitiator,
b—the polymers purified by precipitation are dried under vacuum,
c—next, in the second step, polymerization of the second block consisting of a monomer or a mixture of monomers is performed at the end of the macroinitiator (formed in step a).

Steps b and c are repeated as many times as necessary according to the number of blocks.

One process that may be used in some embodiments is the reverse ATRP polymerization process, which should not be confused with the standard ATRP process described above.

In this reverse ATRP process, the initiation is performed in a conventional manner with an initiator capable of giving radicals, for instance azobisisobutyronitrile (AIBN) or a peroxide (rather than using a specific initiator).

The presence of metal halides, such as CuBr₂ and of a ligand makes it possible to control the polymerization by reversibly scavenging the radicals formed.

The ligands may be, for instance, amine-based molecules, in particular tris(dimethylaminoethyl)amine (Me₆TREN). Good control of the polymerization is observed in particular with the AIBN/CuBr₂/Me₆TREN system.

In another embodiment, the process is the “RAFT” polymerization process.

### Second Mode

The block polymers disclosed herein may also be obtained by using the standard free-radical polymerization technique, by adding the monomers sequentially. In this case, only control of the nature of the blocks is possible (no control of the masses).

This involves polymerizing, in a first stage, a monomer M1 in a polymerization reactor; kinetically monitoring its consumption over time, and then, when M1 is approximately 95% consumed, introducing a new monomer M2 into the polymerization reactor.

A polymer of block structure of M1-M2 type is thus obtained.

More specifically, there is described a first process for preparing a copolymer as described above comprising at least one block A and at least one block B and optionally at least one other block different from the blocks A and B, in which the polymerization is performed via the reverse atom transfer radical polymerization technique (reverse ATRP) comprising the following steps:
a) the monomer(s) from which the block A is prepared is(are) polymerized in the presence of a chain-transfer agent, such as a transition metal halide, a free-radical initiator and a ligand, and in the presence or absence of a solvent, by means of which a macroinitiator or functional precursor capable of initiating a polymerization since it comprises the chain-transfer agent function at its ends is obtained;
b) the monomer(s) from which the block B is prepared is (are) polymerized at the end of the said macroinitiator or precursor in the presence of a chain-transfer agent such as a transition metal halide and a ligand, and in the presence or absence of a solvent, by means of which a diblock copolymer of structure A-b-B is obtained;
c) step b) is optionally repeated with the monomer(s) from which the block or the various other blocks of blocks A and B is (are) prepared, by means of which a triblock or multiblock copolymer is obtained.

It is possible to use difunctional initiators to obtain symmetrical triblock copolymers.

In one embodiment, the chain-transfer agents are chosen from halides of metals in the highest possible oxidation state, such as from CuBr₂, CuCl₂, FeCl₂P(phenyl)₃, FeCl₃ and RuCl₂P(phenyl)₃.

In one embodiment, the free-radical polymerization initiators are chosen from:
azo compounds, such as 2,2′-azobisisobutyronitrile (AIBN), 2,2′-azobis(2-butanenitrile), 4,4′-azobis(4-pentanoic acid), 1,1′-azobis(cyclohexanecarbonitrile), 2-(t-butylazo)-2-cyanopropane, 2,2′-azobis[2-methyl-N-(1,1)-bis(hydroxymethyl)-2-hydroxyethyl]-propionamide, 2,2′-azobis(2-methyl-N-hydroxyethyl)propionamide, 2,2′-azobis(N,N′-dimethyleneisobutyramidine) dichloride, 2,2′-azobis(2-amidinopropane) dichloride, 2,2′-azobis(N,N′-dimethyleneisobutyramide), 2,2′-azobis(2-methyl-N-[1,1-bis(hydroxy-methyl)-2-hydroxyethyl]propionamide), 2,2′-azobis(2-methyl-N-[1,1-bis(hydroxymethyl)ethyl]propionamide), 2,2′-azobis(2-methyl-N-(2-hydroxyethyl)propionamide), 2,2′-azobis(isobutyramide) dihydrate;
hydrogen peroxides, such as tert-butyl hydroperoxide, cumene hydroperoxide, t-butyl peroxyacetate, t-butyl peroxybenzoate, t-butyl peroxyoctoate, t-butyl peroxy-neodecanoate, t-butyl peroxyisobutarate, lauroyl peroxide, t-amyl peroxypivalate, t-butyl peroxypivalate, dicumyl peroxide, benzoyl peroxide;
alkaline persulfates, such as potassium persulfate or ammonium persulfate;
redox systems comprising combinations such as:
   mixtures of hydrogen peroxide, alkyl peroxide, peresters, percarbonates and the like, and any iron salts, titanous salts, zinc formaldehyde sulfoxylate or sodium formaldehyde sulfoxylate, and reducing sugars;
   alkali metal or ammonium persulfates, perborate or perchlorate in combination with an alkali metal bisulfite, such as sodium metabisulfite, and reducing sugars,
   alkali metal persulfates in combination with an arylphosphinic acid, such as benzenephosphonic acid and the like, and reducing sugars.

In one embodiment, the ligands are chosen from tetradentate ligands, such as 1,1,4,7,10,10-hemamethyltriethylenetetramine (HMTETA), 1,4,8,11-tetraazacyclotetradecane (cyclame), 1,4,8,11-tetramethyl-1,4,8,11-tetraazacyclotetradecane (Me₄ cyclame) and tris(dimethylaminoethyl)amine (Me₆TREN); hexadentate ligands, such as tetrakis(2-pyridyl)pyrazine (TPPY), N,N,N′,N′-tetrakis(2-pyridylmethyl)ethylenediamine (TPMEDA) and triphenylphosphine (TPP).

In one embodiment, the solvents are chosen from dioxane, tetrahydrofuran (THF), N-methylpyrrolidone and water, and mixtures thereof.

The chain-transfer agents, initiators, ligands and solvents may be identical or different in step a) and step b), for example, they may be identical. In one embodiment, in the first and second steps a) and b), the chain-transfer agent is CuBr₂, the initiator is AIBN, the ligand is Me₆TREN and the solvent is dioxane.

This first process can make it possible to prepare any of the copolymers of the present disclosure, but it applies particularly to the preparation of a copolymer in which block A is a homopolymer derived from a monomer chosen from N-vinylpyrrolidone and N-vinylcaprolactam, and block B is a homopolymer derived from a monomer chosen from methyl acrylate, t-butyl acrylate and methyl methacrylate.

There is also described a second process for preparing a copolymer as described above comprising at least one block A and at least one block B and optionally at least one other block different from the blocks A and B, in which the polymerization is performed via the RAFT “Reversible Addition-Fragmentation Chain Transfer” polymerization technique including the following two steps:
a) the monomer(s) from which block A is prepared is (are) polymerized in the presence of a chain-transfer agent and an initiator, in a solvent or otherwise, by means of which a macroinitiator or precursor comprising the chain-transfer agent function at its ends is obtained;
b) the monomer(s) from which block B is prepared is (are) polymerized at the end of the said macroinitiator or precursor, in the presence of an initiator, in a solvent or otherwise, by means of which a copolymer A-b-B is obtained;
c) step (b) is optionally repeated with the monomer(s) from which the other block(s); different from blocks A and B is (are) prepared, by means of which a triblock or multiblock copolymer is obtained.

In one embodiment, the chain-transfer agents are chosen from dithioesters (—C(S)S—) such as dithiobenzoates, dithiocarbamates (—NC(S)S—) and dithiocarbonates (—OC(S)S—) (xanthates).

For example, the chain-transfer agents are chosen from diethyl malonate diphenyl dithiocarbamate (DPCM), fluoroethyl acetate diphenyldithiocarbamate (DPFEM) and the xanthate of formula C₂H₅OC(S)SCH(CH₃)COOCH₃.

In one embodiment, the initiators are chosen from the compounds already mentioned above for the first process.

In one embodiment, the solvents are chosen from dioxane, tetrahydrofuran, N-methylpyrrolidone and water, or mixtures thereof.

The initiators, chain-transfer agents and solvents may be identical or different in step a) and step b). In one embodiment, they are identical.

Also, in the first and the second steps a) and b), the initiator is, for example, AIBN, the chain-transfer agent is, for example, diethyl malonate diphenyldithiocarbamate, (DPCM), fluoroethyl acetate diphenyldithiocarbamate (DPFEM) or the xanthate of formula C₂H₅OC(S)SCH(CH₃)COOCH₃ according to the nature of the monomers to be polymerized, and the solvent is, for example, dioxane.

This second process can make it possible to prepare any one of the copolymers of the present disclosure, but it applies particularly to the preparation of a copolymer in which block A is a homopolymer derived from a monomer chosen from N-vinylpyrrolidone and N-vinylcaprolactam, and block B is a homopolymer derived from a monomer chosen from methyl acrylate, t-butyl acrylate and methyl methacrylate.

The present disclosure also relates to cosmetic or pharmaceutical compositions comprising the copolymer of specific structure as has been described above.

Generally, these compositions contain from 0.1% to 60% by weight such as from 0.5% to 50% by weight and further such as from 1% to 40% by weight of the copolymer according to the present disclosure.

These cosmetic compositions, according to the present disclosure, comprise, besides the said polymers, a physiologically acceptable medium, i.e. a medium that is compatible with keratin materials, for instance the skin, the hair, the eyelashes, the eyebrows and the nails.

In general, it should be considered that the whole composition is physiologically acceptable.

The said physiologically acceptable medium generally comprises a suitable physiologically acceptable solvent, in which the copolymer according to the present disclosure is present in dissolved or dispersed form.

The composition may thus comprise, as solvent forming a hydrophilic phase, water or a mixture of water and of hydrophilic organic solvent(s), for instance alcohols such as linear or branched lower monoalcohols containing from 2 to 5 carbon atoms, for instance ethanol, isopropanol or n-propanol, and polyols, for instance glycerol, diglycerol, propylene glycol, sorbitol, pentylene glycol and polyethylene glycols. The hydrophilic phase may also contain C₂ ethers.

The water or the mixture of water and of hydrophilic organic solvents may be present in the composition disclosed herein in a content ranging from 0% to 90% (such as from 0.1% to 90%) by weight or for example from 0% to 60% by weight (such as 0.1% to 60% by weight) relative to the total weight of the composition.

The composition may also comprise a fatty phase, comprising fatty substances that are liquid at room temperature (in general 25° C.) and/or fatty substances that are solid at room temperature, such as waxes, pasty fatty substances and gums, and mixtures thereof. These fatty substances may be of animal, plant, mineral or synthetic origin. This fatty phase may also contain lipophilic organic solvents.

As fatty substances that are liquid at room temperature, often known as oils, which may be used in the present disclosure, mention may be made of: hydrocarbon-based oils of animal origin such as perhydrosqualene; hydrocarbon-based plant oils such as liquid triglycerides of fatty acids containing from 4 to 10 carbon atoms, for instance heptanoic or octanoic acid triglyceride, or alternatively sunflower oil, corn oil, soybean oil, grape seed oil, sesame seed oil, apricot oil, macadamia oil, castor oil, avocado oil, caprylic/capric acid triglycerides, jojoba oil and shea butter oil; linear or branched hydrocarbons of mineral or synthetic origin such as liquid paraffins and derivatives thereof, petroleum jelly, polydecenes and hydrogenated polyisobutene such as parleam; synthetic esters and synthetic ethers, especially of fatty acids, such as, for example, Purcellin oil, isopropyl myristate, 2-ethylhexyl palmitate, 2-octyldodecyl stearate, 2-octyidodecyl erucate and isostearyl isostearate; hydroxylated esters, for instance isostearyl lactate, octyl hydroxystearate, octyldodecyl hydroxystearate, diisostearyl malate, triisocetyl citrate, and fatty alkyl heptanoates, octanoates and decanoates; polyol esters, for instance propylene glycol dioctanoate, neopentyl glycol diheptanoate or diethylene glycol diisononanoate; and pentaerythritol esters; fatty alcohols containing from 12 to 26 carbon atoms, for instance octyldodecanol, 2-butyloctanol, 2-hexyldecanol, 2-undecylpentadecanol and oleyl alcohol; partially hydrocarbon-based or silicone-based fluoro oils; silicone oils, for instance linear or cyclic, volatile or non-volatile polymethylsiloxanes (PDMSs) that are liquid or pasty at room temperature, for instance cyclomethicones, dimethicones, optionally comprising a phenyl group, for instance phenyl trimethicones, phenyltrimethylsiloxydiphenyl siloxanes, diphenylmethyldimethyltrisiloxanes, diphenyl dimethicones, phenyl dimethicones and polymethylphenylsiloxanes; mixtures thereof.

These oils may be present in a content ranging from 0.01% to 90%, such as from 0.1% to 85% by weight, relative to the total weight of the composition.

The composition according to the present disclosure may also comprise at least one organic solvent that is cosmetically acceptable (acceptable tolerability, toxicology and feel). These solvents may generally be present in an amount ranging from 0% to 90%, such as from 0.1% to 90% and further such as from 10% to 90% by weight, and even further such as from 30% to 90%, relative to the total weight of the composition.

As solvents that may be used in the composition of the present disclosure, mention may be made of acetic acid esters, for instance methyl acetate, ethyl acetate, butyl acetate, amyl acetate, 2-methoxyethyl acetate or isopropyl acetate; ketones, for instance methyl ethyl ketone or methyl isobutyl ketone; hydrocarbons, for instance toluene, xylene, hexane or heptane; aldehydes containing from 5 to 10 carbon atoms; ethers containing at least 3 carbon atoms; and mixtures thereof.

The waxes may be hydrocarbon-based waxes, fluoro waxes and/or silicone waxes and may be of plant, mineral, animal and/or synthetic origin. In particular, the waxes have a melting point of greater than 25° C., such as greater than 45° C.

As waxes that may be used in the composition of the present disclosure, mention may be made of beeswax, carnauba wax or candelilla wax, paraffin, microcrystalline waxes, ceresin or ozokerite; synthetic waxes, for instance polyethylene waxes or Fischer-Tropsch waxes, or silicone waxes, for instance alkyl dimethicones or alkoxy dimethicones containing from 16 to 45 carbon atoms.

The gums are generally polydimethylsiloxanes (PDMSs) of high molecular weight or cellulose gums or polysaccharides and the pasty substances are generally hydrocarbon-based compounds, for instance lanolins and derivatives thereof, or PDMSs.

The nature and amount of the solid substances depend on the desired mechanical properties and textures. As a guide, the composition may contain from 0 to 50% by weight, such as from 1% to 30% by weight of waxes relative to the total weight of the composition.

The polymer may be combined with at least one auxiliary film-forming agent. Such a film-forming agent may be chosen from any compound known to those skilled in the art as being capable of fulfilling the desired function, and may be chosen for example from plasticizers and coalescers.

The physiologically acceptable medium of the composition according to the present disclosure may also comprise at least one dyestuff chosen from water-soluble dyes and pulverulent dyestuffs, for instance pigments, nacres and flakes that are well known to those skilled in the art. The dyestuffs may be present in the composition in an amount ranging from 0.01% to 50% by weight, such as from 0.01% to 30% by weight, relative to the weight of the composition.

The term “pigments” as used herein should be understood as meaning white or colored, mineral or organic particles of any form, which are insoluble in the physiological medium and which are intended to color the composition.

The term “nacres” as used herein should be understood as meaning iridescent particles of any form, produced for example in the shell of certain molluscs, or alternatively synthesized.

The pigments may be white or colored, and mineral and/or organic. Among the mineral pigments that may be mentioned are titanium dioxide, optionally surface-treated, zirconium oxide or cerium oxide, and also zinc oxide, iron oxide (black, yellow or red) or chromium oxide, manganese violet, ultramarine blue, chromium hydrate and ferric blue, and metal powders, for instance aluminium powder or copper powder.

Among the organic pigments that may be mentioned are carbon black, pigments of D & C type, and lakes (lacquers) based on cochineal carmine or on barium, strontium, calcium or aluminium.

The nacreous pigments may be chosen from white nacreous pigments such as titanium-coated mica or bismuth oxychloride-coated mica, coloured nacreous pigments such as titanium mica coated with iron oxides, titanium mica coated for instance with ferric blue or with chromium oxide, titanium mica coated with an organic pigment of the abovementioned type and also nacreous pigments based on bismuth oxychloride.

The water-soluble dyes are, for example, beetroot juice or methylene blue.

The composition according to the present disclosure may also comprise at least one filler, present, for example, in an amount ranging from 0.01% to 50% by weight, such as from 0.01% to 30% by weight, relative to the total weight of the composition. As used herein, the term “fillers” should be understood as meaning colorless or white, mineral or synthetic particles of any form, which are insoluble in the medium of the composition, irrespective of the temperature at which the composition is manufactured. These fillers may serve to modify the rheology or the texture of the composition.

The fillers may be mineral or organic of any form, platelet, spherical or oblong, irrespective of the crystallographic form (for example lamellar, cubic, hexagonal, orthorhombic, etc.). Mention may be made of talc, mica, silica, kaolin, polyamide powder (Nylon®) (Orgasol® from Atochem), poly-β-alanine powder and polyethylene powder, tetrafluoroethylene polymer (Teflon®) powders, lauroyllysine, starch, boron nitride, hollow polymer microspheres such as those of polyvinylidene chloride/acrylonitrile, for instance Expancel® (Nobel Industrie) or of acrylic acid copolymers (Polytrap® from the company Dow Corning) and silicone resin microbeads (for example Tospearls® from Toshiba), polyorganosiloxane elastomer particles, precipitated calcium carbonate, magnesium carbonate, magnesium hydrocarbonate, hydroxyapatite, hollow silica microspheres (Silica Beads® from Maprecos), glass or ceramic microcapsules, metal soaps derived from organic carboxylic acids containing from 8 to 22 carbon atoms such as from 12 to 18 carbon atoms, for example zinc stearate, magnesium stearate, lithium stearate, zinc laurate or magnesium myristate.

The composition according to the present disclosure may also contain ingredients commonly used in cosmetics, such as vitamins, thickeners, trace elements, softeners, sequestering agents, fragrances, acidifying or basifying agents, preserving agents, sunscreens, surfactants, antioxidants, agents for preventing hair loss, antidandruff agents, propellants, and film-forming or non-film-forming water-soluble or liposoluble polymers or polymers dispersed in water or in a fatty phase, or mixtures thereof.

Needless to say, a person skilled in the art will take care to select this or these optional additional compound(s), and/or the amount thereof, such that the advantageous properties of the corresponding composition according to the present disclosure are not, or are not substantially, adversely affected by the envisaged addition.

The composition according to the present disclosure may for example be in the form of a suspension, a dispersion, a solution, a gel, an emulsion, especially an oil-in-water (O/W) or water-in-oil (W/O) emulsion, or a multiple emulsion (W/O/W or polyol/O/w or O/W/O emulsion), in the form of a cream, a paste, a mousse, a dispersion of vesicles, especially of ionic or nonionic lipids, a two-phase or multi-phase lotion, a spray, a powder, a paste, such as a soft paste (for example, a paste having a dynamic viscosity at 25° C. of about from 0.1 to 40 Pa·s at a shear rate of 200 s⁻¹ after measurement for 10 minutes in cone/plate geometry). The composition may be anhydrous, for example it may be an anhydrous paste.

A person skilled in the art may select the appropriate presentation form, and also the method for preparing it, on the basis of his or her general knowledge, taking into account firstly the nature of the constituents used, especially their solubility in the support, and secondly the intended use of the composition.

The composition according to the present disclosure may be a makeup composition, for instance complexion products (foundations), rouges, eye shadows, lip products, concealer products, blushers, mascaras, eyeliners, eyebrow makeup products, lip pencils, eye pencils, nail products, such as nail varnishes, body makeup products or hair makeup products (hair lacquer or mascara).

The composition according to the present disclosure may also be a hair product, e.g., for holding the hairstyle or for shaping the hair. The hair compositions may, for example, be shampoos, hairsetting gels or lotions, blow-drying lotions, or fixing and styling compositions such as lacquers or sprays.

When the polymer according to the present disclosure is used in hair products, such as products for holding the hairstyle or for shaping the hair, it makes it possible to avoid “flaking” powdering.

The solutions may be packaged in various forms: for example as gels or lotions, and in vaporizers, in pump-dispenser bottles or in aerosol containers in order to allow the composition to be applied in vaporized form or in the form of a mousse. Such packaging forms are indicated, for example, when it is desired to obtain a spray or a mousse for fixing or treating the hair.

It has been seen hereinabove that the polymer according to the present disclosure may be suitable for use under such conditions since it is highly compatible with the propellants used in aerosol containers, for example. The propellant(s) may be chosen from dimethyl ether, C₃₋₅ alkanes, such as propane, n-butane and isobutane; 1,1-difluoroethane, mixtures of dimethyl ether and of C₃₋₅ alkanes, and mixtures of 1,1-difluoroethane and of dimethyl ether and/or of C₃₋₅ alkanes.

The invention will now be described with reference to the following examples, which are given as non-limiting illustrations.

### EXAMPLES

In the examples below, polymers according to the invention were prepared via the reverse atom-transfer radical polymerization technique, also known as reverse ATRP, or via the RAFT (reverse addition-fragmentation chain transfer) polymerization technique.

In general, the formation of blocks in the case of the reverse ATRP process and of the RAFT process takes place in several steps.

Thus, in the case of a diblock copolymer, the process will be performed, for example, in the following manner:
a/ first step: polymerization of the first monomer to form the macroinitiator or precursor;
b/ second step: polymerization of the second monomer at the end of the macroinitiator, to form the diblock.

Between the two steps, a purification step may be necessary, for example in the case of the reverse ATRP polymerization.

The general procedure used to perform the examples will first be described, for each of the processes: process 1 (reverse ATRP polymerization) and process 2 (RAFT polymerization).

### 1. Process 1: Reverse ATRP

The reverse ATRP process is performed, in one embodiment, with tris(dimethylaminoethyl)amine (TREN Me₆) as ligand, using CuBr₂ and employing AIBN as initiator.

### 1.1 Starting Materials

The ethylenic monomer containing a lactam ring, such as N-vinylpyrrolidone (VP), obtained from the company Aldrich®, is distilled under vacuum and stored under nitrogen at 0° C. before use.
The other monomers, such as tert-butyl acrylate, methyl methacrylate and methyl acrylate, obtained from the company Aldrich®, are dried over calcium hydride and distilled under vacuum.
The initiator azobisisobutyronitrile (AIBN) is recrystallized from methanol.
CuBr₂ (99.9%), CuBr and copper powder (99%) are obtained from the company Aldrich® and used as supplied.
The tris(dimethylaminoethyl)amine (TREN Me₆) is synthesized according to the procedures described in the literature, for example in the Matyjaszewski document ACS Symp. Ser. 2000; 760; 207.
All the solvents: THF, dioxane and DMF, are dried by distillation over CaH₂ before use.

### 1.2. Polymerization

The general procedure is as follows:

### 1.2.1. First Step

### Formation of the Macroinitiator or Precursor

CuBr₂ and Me₆ TREN are placed in a round-bottomed flask and dioxane is added thereto.

The solution is stirred for 30 minutes at 25° C.

The monomer is added, followed by addition of the initiator AIBN.

The system is subjected to three cycles of vacuum-drying/argon. The solution is then heated in a thermostatically-regulated bath.

The viscosity increases. After reaction for the desired time, the system is cooled to room temperature.

The macroinitiator polymer is separated out by precipitation: it is diluted in chloroform and then precipitated from diethyl ether; this operation is repeated twice. The copper complex is separated from the polymer dissolved in the chloroform by passing through a neutral column. The polymer solution is then evaporated.

### 1.2.2. Second Step

### Polymerization of the Second Block at the End of the Macroinitiator

The macroinitiator obtained in the first step is added to a solution of CuBr and Me₆TREN in dioxane under an inert atmosphere of nitrogen.

The required amount of monomer(s) constituting the second block is added. The solution is heated. After the desired time, the system is cooled to room temperature.

The polymer is separated out by precipitation: it is diluted in chloroform and then precipitated from diethyl ether; this operation is repeated twice. The copper complex is separated from the polymer dissolved in the chloroform by passing through a neutral column. The polymer solution is evaporated.

The polymer is dried.

The isolated polymer is generally a white powder.

### 2. Process 2: RAFT

Ethylenic monomers containing a lactam ring, for example vinyllactams, are polymerized with the following compounds as chain-transfer agents:
diethyl malonate diphenyldithiocarbamate (DPCM):

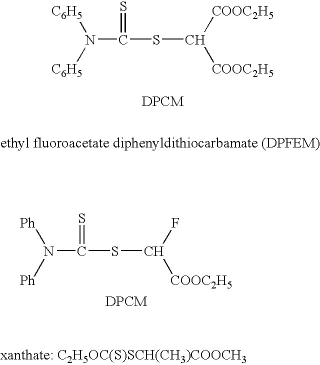

### Starting Materials

The ethylenic monomer containing a lactam ring, such as N-vinylpyrrolidone (VP) or N-vinylcaprolactam (VCap) is obtained from the company Aldrich® and is distilled under vacuum before use.
The other monomers, such as tert-butyl acrylate, methyl acrylate and methyl methacrylate, are obtained from the company Aldrich® and are dried over calcium hydride and distilled under vacuum.
The AIBN is obtained from the company Aldrich® and is recrystallized from methanol.
The diphenylamine, the CS₂ and the bromodiethyl malonate are obtained from the company Aldrich® and are used as received.
The solvents such as dioxane are dried by distillation over CaH₂ before use.

### 2.1. Synthesis of the Chain-Transfer Agent

### 2.1.1. Synthesis of Diethyl Malonate Diphenyldithiocarbamate (DPCM)

Diethyl malonate diphenyldithiocarbamate (DPCM) is synthesized from diphenylamine and bromodiethyl malonate in the following manner:

1.69 g of diphenylamine (10 mmol) in 10 ml of DMSO and 5 ml of THF are added at 0° C. to a solution of 0.625 g of NaH (purified by washing with dry hexane) in 5 ml of THF (dry). The reaction mixture is stirred for 1.5 hours to give a clear green solution. 1.2 eq of CS₂ (1.42 ml, 1.2 mmol) are added to this solution and the mixture is stirred for 30 minutes at 0° C. to obtain an orange-yellow solution of the sodium salt of diphenyl-dithiocarbamate.

Bromodiethyl malonate (10 mmol) is added to the above solution at −20° C. and the temperature of the reaction mixture is brought slowly up to room temperature. After stirring for 2 hours at room temperature, the reaction mixture is treated with water and extracted with ether. The ether layer is dried over MgSO₄ and concentrated.

Yield: 51%

The purity of the product was checked by NMR.

### 2.1.2. Synthesis of Fluoroethyl Acetate Diphenyldithiocarbamate (DPFEA)

Sodium hydride (7 mmol/0.168 g, 1.3 eq in 5 ml of THF) is placed in a flame-dried flask and stirred at 0° C. Diphenylamine (5.4 mmol, 1 eq) in 5 ml of THF and 10 ml of DMSO is added dropwise to this mixture and stirred for 1 hour. Carbon disulfide (2.3 eq) is added to the solution at 0° C., and stirring is continued for a further half an hour. The temperature of the solution is reduced to 18° C. and one equivalent of fluoroethyl acetate is added. After addition, the reaction mixture is warmed slowly to room temperature and stirred for half an hour at room temperature. The product obtained is hydrolysed by adding water and the organic layer is extracted with ether. The ether extract is concentrated to give yellow crystals of (DPFEA) and the purity of the products checked by NMR analysis.

### 2.2. Polymerization

2.2.1. The general procedure for the polymerization is the following, whether the chain-transfer agent is diethyl malonate diphenyldithiocarbamate or fluoroethyl acetate dithiocarbamate.

In the text hereinbelow, vinylpyrrolidone is mentioned, but the preparation process can be generalized to any ethylenic monomer containing a lactam ring.

### First Step

### Preparation of the Polyvinylpyrrolidone Macroinitiator

The polymerization of vinylpyrrolidone (VP) using diethyl malonate diphenyldithiocarbamate as RAFT reagent is performed using AIBN as initiator.

In a typical experiment, VP, the diethyl malonate diphenyldithiocarbamate (chain-transfer agent/monomer ratio=1/100), the AIBN (10% of the chain-transfer agent) and dioxane (ratio of 1/1 by volume relative to the monomer) are placed in a Schlenk tube. The reaction mixture is degassed by three freeze-pump-thaw cycles and then sealed hermetically under vacuum and heated in a bath at a constant temperature of 80° C.

The “active” macroinitiator, which may be represented by ‘X-PVP-X with X=(phenyl)₂NC(S)S—, and X′=—CH(COOC₂H₅)₂, obtained is purified by repeated precipitations from ethyl ether and is dried under vacuum.

### Second Step

### Polymerization of the Second Block at the End of the “Active” Macroinitiator

The polymerization of the second monomer (which is, for example, methyl methacrylate) takes place in the presence of the PVP macroinitiator above, in 1.5 ml of dioxane and in the presence of AIBN (0.1 mol % relative to the total amount of monomer and of macroinitiator).

The reaction mixture is subjected to three freeze-pump-thaw cycles and heated in an oil bath at 80° C. with stirring for 20 hours.

Once the reaction is complete, the reaction mixture is dissolved in dichloromethane (minimum amount required for dissolution) and is precipitated from ether.

The cloudy ether solution is filtered and concentrated by flash evaporation, and is added to pentane; the precipitate obtained is dried under vacuum at about 70° C. for 8 hours.

2.2.2. As a variant, the polymerization may be performed in the following manner: the synthesis of a poly(methyl methacrylate) macroinitiator is first performed, for example, and the polymerization of the vinylpyrrolidone block is then performed at the end of this macroinitiator. The same procedure as in paragraph 2.2.1. is followed.

2.2.3. Polymerization Using a Xanthate as Chain-Transfer Agent: Synthesis without an Intermediate Purification Step (“One-Pot Synthesis”)

N-Vinylpyrrolidone (4 ml, 3.74 eq; 10⁻² mol) (the procedure may also be applied to another ethylenic monomer containing a lactam ring), the chain-transfer agent (above xanthate, 0.0839 g, chain-transfer agent/monomer ratio=about 1/100 on a molar basis) and dioxane (4 ml) are placed in a predried round-bottomed flask and AIBN (0.0061 g, 10 mol % of the chain-transfer agent) is added thereto under nitrogen.

The reaction mixture is subjected to three freeze-pump-thaw cycles and is heated in an oil bath thermostatically maintained at 80° C., with stirring. The reaction is stopped after 14 hours by cooling in liquid nitrogen.

The second monomer, for example buthyl acrylate (3 ml), and dioxane (3 ml) are added to this reaction mixture at room temperature under nitrogen. The reaction mixture is again subjected to three freeze-pump-thaw cycles and is heated at 80° C. for a further 24 hours.

Once the reaction is complete, the reaction mixture is dissolved in dichloromethane (minimum amount required for dissolution) and is precipitated from ether. The cloudy ether solution is filtered and concentrated by flash evaporation, it is added to pentane and the precipitate obtained is dried under vacuum at about 70° C. for 8 hours.

### Characterizations

The conversion is measured by weighing the polymer.
The mass of the polyvinyllactam macroinitiator, for example PVP, is determined by gas chromatography GC (Varian 9012®D machine) with G4000 G3000 G2500 TSK® gel columns, equipped with a Varian® RI 4 infrared detector. The eluent is an 80/20 water/methanol mixture containing 0.1 M sodium nitrate. The flow rate of the eluent is 0.5 ml/minute. The calibration is performed with poly(ethylene oxide) standards.

The mass measurement is therefore only a comparative measurement and the values do not represent the actual molecular masses. However, the results may be used to note the trend in the change of the molecular masses and also to determine the mass dispersity of the chains.
The mass of the water-insoluble macroinitiator (polymethyl methacrylate, for example) is determined by GC (Varian® 9012) with G4000 G3000 G2500 TSK® gel columns, equipped with a Varian® RI 4 infrared detector. The eluent is THF. The standard is polystyrene.
The overall mass of the copolymer is determined by GC chromatography in solvent phase (Varian® 9012) with G4000 G3000 G2500 TSK® gel columns, equipped with a Varian® RI 4 infrared detector. The eluent is THF. The standard is polystyrene.
The proportion of the various blocks is determined by ¹H NMR (Bruker®, 200 MHz), by determining the ratio of the areas of the peaks corresponding to the monomers of the various blocks (i.e., on the one hand MMA and on the other hand VP, for example).
   The overall mass (NMR) of the copolymer is deduced with these values and the mass of the macroinitiator determined above.

After having described above the general procedure for the synthesis of the copolymers according to the present disclosure, examples concerning the preparation of specific copolymers in accordance with the present disclosure (Examples 1, 2 and 3) and also a comparative example are given below.

### Comparative Example

In this example, a copolymer (not in accordance with the present disclosure) comprising a polyvinylpyrrolidone block and a poly(methyl methacrylate) block: (PVP) 80%-b-(PMMA) 20% was prepared according to process 1 (reverse ATRP) described above.

### First Step

### Synthesis of the Polyvinylpyrrolidone-Br Macroinitiator: PVP-Br

CuBr₂ and Me₆TREN were placed in a 50 ml round-bottomed flask and 5 ml of dioxane were added.

Vinylpyrrolidone was then added, followed by addition of AIBN (azobisisobutyronitrile).

The dioxane/monomer ratio was 1/1 by volume.

The AlBN/CuBr₂/CuO/Me₆TREN proportions were: 1/1.5/0.15/3.

The reaction was performed at a temperature of 80° C. for 3 hours 45 minutes. The conversion reached 84%.

### Second Step

Synthesis of the copolymer: Polymerization of Methyl Methacrylate (MMA) at the End of the Polyvinylpyrrolidone Macroinitiator (PVP-Br)

The PVP macroinitiator (PVP-Br) was added to CuBr ( 1/1.5 ratio with respect to PVP-Br) and Me₆TREN (⅓ with respect to CuBr) in dioxane under a nitrogen atmosphere.

Polyvinylpyrrolidone macroinitiator PVP-Br: 0.026 mM.

Methyl methacrylate (MMA): 3.87 mM.

The reaction was performed at a temperature of 100° C. for 3 hours 45 minutes.

The conversion was 60%.

The characteristics of the macroinitiator and of the final copolymer are summarized in the following table:

| | | Mn (PVP | | | Mn PMMA |
|---|---|---|---|---|---|
| | | macroinitiator) | % | % PMMA/total | deduced by |
| Copolymer | VP/AIBN | (GC) | PVP/totalweight | weight | NMR |
| (PVP) | 1/0.25 | 38 700 | 80.2% | 19.8% | 9600 |
| 80%--b-- | | | | | |
| (PMMA) 20% | | | | | |

Vinylpyrrolidone = VP
Azobisisobutyronitrile = AIBN
Mₙ = number-average molecular mass

The polymer obtained was dissolved in ethanol to a proportion of 6% by weight.

The polymer was spread out and gave a brittle film. It was not possible to obtain a continuous film.

### Example 1

In this example, a copolymer (in accordance with the present disclosure) comprising a polyvinylpyrrolidone (PVP) block, and a poly(methyl acrylate) (pMeA) block was prepared: (PVP) 66%-b-polymethyl acrylate (PMeA) 34%, according to the process described in paragraph 2.2.3. above.

The N-vinylpyrrolidone (VP) (3.74×10⁻² mol i.e. 4.15 g), the chain-transfer agent (xanthate described above, 0.0839 g, (chain-transfer agent/monomer ratio: about 1/100 in moles) and dioxane (4 ml) were placed in a predried flask, and AIBN (0.0061 g, 10 mol % relative to the chain-transfer agent) was added to the flask under nitrogen.

This reaction mixture was subjected to three freeze-pump-thaw cycles and was heated in an oil bath thermostatically maintained at 80° C., with stirring.

The reaction was stopped after 14 hours by cooling in liquid nitrogen.

The second monomer (methyl methacrylate) (3 ml) and dioxane (3 ml) were added to this reaction mixture at room temperature, under nitrogen. The reaction mixture was again subjected to three freeze-pump-thaw cycles and heated at 80° C. for a further 24 hours.

Once the reaction was complete, the reaction mixture was dissolved in dichloromethane (minimum amount required for dissolution) and precipitated from ether.

The cloudy ether solution was filtered and concentrated by flash evaporation, and was added to pentane; the precipitate obtained was dried under vacuum at about 70° C. for 8 hours, and was characterized by GPC and NMR.

The characteristics of the reagents and of the process are summarized below.

Chain-transfer agent: xanthate C₂H₅OC(S)SCH(CH₃)COOCH₃.

Molar ratio of xanthate/VP monomer: 1/100.

AIBN/xanthate chain-transfer agent ratio: 10 mol %.

Monomers/AIBN ratio: 0.1

Reaction temperature: 80° C.

Solvent: dioxane.

The characteristics of the macroinitiator and of the final copolymer are summarized in the following table:

| | | Mn | | % of VP | Molecular masses of the | | | |
|---|---|---|---|---|---|---|---|---|
| | | (macroinitiator | Conversion | by | final copolymer | | | |
| | Conversion | PVP) | of the | weight | (GPC) | | | |
| Copolymer | VP | (GC) | acrylate | (NMR) | Mp | Mn | Mw | PDI |
| PVP | 88% | 12560 | 75% | 34 | 42900 | 20500 | 44770 | 22 |
| 66%- | | | | | | | | |
| b- | | | | | | | | |
| (PMeA) | | | | | | | | |
| 34% | | | | | | | | |

Mn: number-average molecular mass
Mw: weight-average molecular mass
Mp: molecular mass at the peak
PDI: polydispersity index = Mw/Mn
Poly(methyl acrylate): PMeA

### Example 2

In this example, a copolymer (in accordance with the present disclosure) comprising a polyvinylpyrrolidone (PVP) block and a poly(butyl acrylate) (PBUA) block was prepared: (PVP) 60%-b-(PBuA) 40%.

The procedure was the same as in Example 1 (process of paragraph 2.2.3.), only the proportions and the nature of the monomers changed relative to Example 1. The characteristics of the macroinitiator and of the final copolymer are summarized in the following table:

| | | Mn | % of VP | Molecular masses of the final | | | |
|---|---|---|---|---|---|---|---|
| | Conversion | (macroinitiator PVP) | by | copolymer (GPC) | | | |
| Copolymer | VP | (GC) | weight | Mp | Mn | Mw | PDI |
| PVP 60-b- | 69 | 8520 | 60 | 55610 | 34000 | 66700 | 1.97 |
| poly(butyl | | | | | | | |
| acrylate) 40% | | | | | | | |

The polymer was dissolved in ethanol to a proportion of 6% by weight.

It resulted in a non-brittle and non-tacky film.

### Example 3

In this example, a copolymer (in accordance with the present disclosure) comprising a polyvinylcaprolactam (PVCap) block and a poly(butyl acrylate) (PBUA) block was prepared:

(PVCap) 10%-b-(PBuA) 90%.

The synthesis of the copolymer was performed according to the process of paragraph 2.2.2.

### First Step

Synthesis of the “active” poly(vinylcaprolactam) (PVCap) macroinitiator

VCap (1 g, 7.183 mmol), the chain-transfer agent DPFEA (0.0244 g) (chain-transfer agent ═NVCap monomer/50 mol) and the AIBN (20 mol % of the chain-transfer agent) in dioxane were placed in a predried flask and subjected to three freeze-pump-thaw cycles to remove the oxygen present. The reaction mixture was immersed in an oil bath thermostatically maintained at 80° C. and stirred for 17 hours.

The reaction was stopped by freezing in liquid nitrogen and a small amount of the sample was taken for analytical purposes.

### Second Step

### Synthesis of the Copolymer: Polymerization of Butyl Acrylate at the End of the Polyvinylcaprolactam (PVCap) “Active” Macroinitiator

The second monomer (butyl acrylate, 1 ml) and dioxane (1 ml) were added directly to the reaction mixture and the oxygen was removed again via freeze-pump-thaw cycles. The reaction was continued for 24 hours at 60° C. Once the reaction was complete, the reaction mixture was diluted with dichloromethane and the product was precipitated from pentane and dried under vacuum at room temperature.

The characteristics of the macroinitiator and of the final copolymer are summarized in the following table:

| | | | % BuA by | |
|---|---|---|---|---|
| | Mn (PVCap | % BuA in the | weight in the | |
| | macroinitiator) | copolymer | copolymer | Mn PBuA |
| Copolymer | (NMR) | (NMR) | (NMR) | (NMR) |
| PVCap-b-P | 10400 | 88 mol % | 88 | 68900 |
| MMA | | | | |

1-88. (canceled)

89. A cosmetic composition comprising at least one linear block ethylenic copolymer comprising:
at least one block A derived from monomers comprising from 52 to 100% by weight of at least one ethylenic monomer containing a lactam ring, corresponding to formula (II) below: 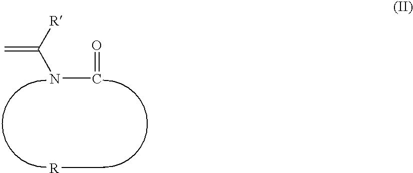
wherein:
R is a group —(CH₂)ₙ—, in which at least one of the carbon atoms is optionally replaced with a nitrogen atom or an oxygen atom, n is an integer chosen from 3 to 12, and at least one of the carbon atoms is optionally substituted with at least one C₁ to C₆ alkyl group;
R′ is chosen from H and a methyl group; and
at least one block B derived from monomers not comprising an ethylenic monomer containing a lactam ring of formula (II), or comprising a minor proportion thereof, the at least one block B having a glass transition temperature Tg ranging from −55° C. to +55° C.;
wherein the monomers from which the at least one block B may be prepared are chosen from: hydroxyethyl methacrylate, isobutyl methacrylate, n-hexyl acrylate, t-butyl acrylate, butyl methacrylate, cyclohexyl acrylate, hydroxyethyl acrylate, ethoxyethyl methacrylate, n-hexyl methacrylate, vinyl butyrate, isobutyl acrylate, methyl vinyl ether, methoxyethyl acrylate, n-butyl acrylate, POE methacrylate (n=8 to 10), and mixtures thereof.

90. A cosmetic composition according to claim 89, wherein the at least one linear block ethylenic copolymer is present in an amount ranging from 0.1% to 60% by weight, relative to the total weight of the composition.

91. A cosmetic composition according to claim 90, wherein the at least one linear block ethylenic copolymer is present in an amount ranging from 0.5% to 50% by weight, relative to the total weight of the composition.

92. A cosmetic composition according to claim 91, wherein the at least one linear block ethylenic copolymer is present in an amount ranging from 1% to 40% by weight, relative to the total weight of the composition.

93. A cosmetic composition according to claim 89, further comprising a physiologically acceptable medium in which the at least one linear block ethylenic copolymer is in dissolved or dispersed form.

94. A cosmetic composition according to claim 93, in which the physiologically acceptable medium comprises at least one solvent suitable for forming a hydrophilic phase, chosen from water and mixtures of water and of at least one hydrophilic organic solvent.

95. A cosmetic composition according to claim 94, wherein the at least one hydrophilic organic solvent is chosen from at least one alcohol.

96. A cosmetic composition according to claim 95, wherein the at least one alcohol is chosen from linear and branched lower monoalcohols containing from 2 to 5 carbon atoms.

97. A cosmetic composition according to claim 96, wherein the linear and branched lower monoalcohols containing from 2 to 5 carbon atoms are chosen from ethanol, isopropanol, n-propanol, and polyols.

98. A cosmetic composition according to claim 97, wherein the polyols are chosen from glycerol, diglycerol, propylene glycol, sorbitol, pentylene glycol and polyethylene glycols.

99. A cosmetic composition according to claim 94, wherein the hydrophilic phase further comprises at least one C₁ to C₂ ether.

100. A cosmetic composition according to claim 93, wherein the physiologically acceptable medium also comprises a fatty phase comprising fatty substances that are liquid or solid at room temperature, and are of animal, plant, mineral or synthetic origin.

101. A cosmetic composition according to claim 89, further comprising at least one cosmetically acceptable organic solvent.

102. A cosmetic composition according to claim 93, wherein the physiologically acceptable medium further comprises at least one auxiliary film-forming agent chosen from plasticizers and coalescers.

103. A cosmetic composition according to claim 89, further comprising at least one dyestuff chosen from water-soluble dyes and pulverulent dyestuffs.

104. A cosmetic composition according to claim 89, further comprising at least one filler.

105. A cosmetic composition according to claim 89, further comprising at least one ingredient chosen from vitamins, thickeners, trace elements, softeners, sequestering agents, fragrances, acidifying or basifying agents, preserving agents, sunscreens, surfactants, antioxidants, agents for preventing hair loss, antidandruff agents, propellants, film-forming and non-film-forming water-soluble and liposoluble polymers and polymers dispersed in water or in a fatty phase.

106. A cosmetic composition according to claim 89, wherein said composition is in a form chosen from a suspension, a dispersion, a solution, a gel, an emulsion, a cream, a mousse, a dispersion of vesicles, a two-phase or multi-phase lotion, a spray, a powder, and a paste.

107. A cosmetic composition according to claim 89, wherein said composition is a hair product.

108. A cosmetic composition according to claim 89, wherein said composition is in the form of a makeup composition.

109. A cosmetic composition according to claim 103, wherein the pulverulent dyestuffs are chosen from pigments, nacres and flakes.

110. A cosmetic composition according to claim 106, wherein the emulsion is chosen from an oil-in-water (O/W) emulsion, a water-in-oil (W/O) emulsion, and a multiple emulsion.

111. A cosmetic composition according to claim 110, wherein the multiple emulsion is chosen from W/O/W, polyol/O/W and O/W/O emulsions.

112. A cosmetic composition according to claim 106, wherein the dispersion of vesicles is chosen from a dispersion of ionic lipids and a dispersion of nonionic lipids.

113. A cosmetic composition according to claim 106, wherein the paste is chosen from a soft paste and an anhydrous paste.

114. A cosmetic composition according to claim 107, wherein the hair product is chosen from shampoos and lacquers.

115. A cosmetic composition according to claim 112, wherein the makeup composition is a nail varnish.

116. A cosmetic composition according to claim 89, wherein n is an integer chosen from 3 to 5, or R is —CH₂—CH₂—NH—.

117. A cosmetic composition according to claim 89, wherein said at least one linear block ethylenic copolymer is a film-forming copolymer.

118. A cosmetic composition according to claim 89, wherein said at least one linear block ethylenic copolymer has an overall number-average molecular mass ranging from 4,000 to 1,000,000.

119. A cosmetic composition according to claim 118, wherein said at least one linear block ethylenic copolymer has an overall number-average molecular mass ranging from 4,000 to 800,000.

120. A cosmetic composition according to claim 89, wherein said linear block ethylenic copolymer has an overall number-average molecular mass ranging from 4000 to 500,000.

121. A cosmetic composition according to claim 89, wherein said at least one block A is present in an amount ranging from 1% to 99% by weight, relative to the total weight of the at least one linear block ethylenic copolymer.

122. A cosmetic composition according to claim 121, wherein said at least one block A is present in an amount ranging from 10% to 95% by weight, relative to the total weight of the at least one linear block ethylenic copolymer.

123. A cosmetic composition according to claim 122, wherein said at least one block A is present in an amount ranging from 20% to 90% by weight, relative to the total weight of the at least one linear block ethylenic copolymer.

124. A cosmetic composition according to claim 89, wherein said at least one block B is present in an amount ranging from 1% to 99% by weight, relative to the total weight of the at least one linear block ethylenic copolymer.

125. A cosmetic composition according to claim 124, wherein said at least one block B is present in an amount ranging from 5% to 90% by weight, relative to the total weight of the at least one linear block ethylenic copolymer.

126. A cosmetic composition according to claim 125, wherein said at least one block B is present in an amount ranging from 10% to 80% by weight, relative to the total weight of the at least one linear block ethylenic copolymer.

127. A cosmetic composition according to claim 89, wherein the number-average molecular mass of each of said at least one block A or B ranges from 2,000 to 1,000,000.

128. A cosmetic composition according to claim 127, wherein the number-average molecular mass of each of said at least one block A or B ranges from 2,000 to 800,000.

129. A cosmetic composition according to claim 128, wherein the number-average molecular mass of each of said at least one block A or B ranges from 2,000 to 500,000.

130. A cosmetic composition according to claim 89, wherein the at least one ethylenic monomer of formula (II) containing a lactam ring is present in the at least one block A in an amount ranging from 55% to 95% by weight, relative to the total weight of the at least one block A.

131. A cosmetic composition according to claim 130, wherein the at least one ethylenic monomer of formula (II) containing a lactam ring is present in the at least one block A in an amount of 90% by weight, relative to the total weight of the at least one block A.

132. A cosmetic composition according to claim 131, wherein the at least one block A is a homopolymer of the at least one ethylenic monomer containing a lactam ring of formula (II).

133. A cosmetic composition according to claim 132, wherein the homopolymer is a homopolymer of an N-vinyllactam of formula (II).

134. A cosmetic composition according to claim 133, wherein the N-vinyllactam of formula (II) is chosen from N-vinylpyrrolidone (n=3), N-vinylpiperidinone (valerolactam) (n=4), N-vinylcaprolactam (n=5), N-vinylimidazolidinone in which R is a —CH₂—CH₂—NH— group, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam and N-vinyl-7-ethyl-2-caprolactam.

135. A cosmetic composition according to claim 134, wherein the at least one block A is an N-vinylpyrrolidone homopolymer.

136. A cosmetic composition according to claim 134, wherein the at least one block A is an N-vinylcaprolactam homopolymer.

137. A cosmetic composition according to claim 89, wherein the at least on block A is a copolymer derived from monomers comprising, in addition to the at least one ethylenic monomer containing a lactam ring of formula (II), at least one other monomer.

138. A cosmetic composition according to claim 137, wherein the at least one block A is a random, alternating, or gradient copolymer.

139. A cosmetic composition according to claim 137, wherein the at least one other monomer of the at least one A block is chosen from monomers for which the glass transition temperature of the corresponding homopolymer is less than or equal to 50° C.

140. A cosmetic composition according to claim 139, wherein the at least one other monomer of the at least one block A is chosen from monomers for which the glass transition temperature of the corresponding homopolymer is less than or equal to 20° C.

141. A cosmetic composition according to claim 140, wherein the at least one other monomer of the at least one block A is chosen from monomers for which the glass transition temperature of the corresponding homopolymer is less than or equal to 0° C.

142. A cosmetic composition according to claim 139, wherein the at least one other monomer of the at least one block A also has a glass transition temperature for the corresponding homopolymer of greater than or equal to −150° C.

143. A cosmetic composition according to claim 139, wherein the at least one other monomer of the at least one block A for which the glass transition temperature of the corresponding homopolymer is less than or equal to 50° C. is chosen from: t-butyl acrylate, vinyl acetate, butyl methacrylate, cyclohexyl acrylate, hydroxyethyl acrylate, methyl acrylate, ethoxyethyl methacrylate, n-hexyl methacrylate, ethyl acrylate, isobutyl acrylate, methyl vinyl ether, methoxyethyl acrylate, n-butyl acrylate, ethylhexyl acrylate, POE methacrylate (n=8 to 10) and isobornyl methacrylate; butylacrylamide, N-isopropylacrylamide, N,N-dimethylacrylamide and N,N-dibutylacrylamide, hexyl acrylate, octyl acrylate, lauryl acrylate, isooctyl acrylate, ethylhexyl methacrylate, octyl methacrylate, lauryl methacrylate, vinyl neononanoate, vinyl neododecanoate; and N-octylacrylamide.

144. A cosmetic composition according to claim 137, wherein at least one of the other monomers of the at least one block A is chosen from hydrophilic monomers.

145. A cosmetic composition according to claim 137, wherein at least one of the other monomers of the at least one block A is chosen from non-hydrophilic monomers.

146. A cosmetic composition according to claim 89, wherein the at least one block A has an overall glass transition temperature ranging from 0 to 250° C.

147. A cosmetic composition according to claim 146, wherein the at least one block A has an overall glass transition temperature ranging from 0 to 220° C.

148. A cosmetic composition according to claim 146, wherein the at least one block A has an overall glass transition temperature ranging from 5 to 200° C.

149. A cosmetic composition according to claim 89, wherein the at least one block A is chosen from hydrophilic blocks.

150. A cosmetic composition according to claim 89, wherein the at least one block B has a glass transition temperature Tg ranging from −50° C. to +50° C.

151. A cosmetic composition according to claim 89, wherein the at least one block B has a glass transition temperature Tg ranging from −50° C. to +45° C.

152. A cosmetic composition according to claim 89, wherein the at least one block B is a homopolymer or copolymer.

153. A cosmetic composition according to claim 152, wherein the at least one block B is formed from a copolymer comprising
a first monomer for which the Tg of the corresponding homopolymer is in the range from +20° C. to +250° C., and
a second monomer for which the Tg of the corresponding homopolymer is in the range from −200° C. to +20° C.

154. A cosmetic composition according to claim 151, wherein the at least one block B is a homopolymer comprising one type of monomer for which the Tg of the corresponding homopolymer is in the range from −55° C. to +55° C.

155. A cosmetic composition according to claim 154, wherein the at least one block B is a homopolymer comprising only one type of monomer for which the Tg of the corresponding homopolymer is in the range from −50° C. to +50° C.

156. A cosmetic composition according to claim 155 wherein the at least one block B is a homopolymer comprising only one type of monomer for which the Tg of the corresponding homopolymer is in the range from −50° C. to +45° C.

157. A cosmetic composition according to claim 89, wherein at least one of the monomers constituting the at least one block B is chosen from hydrophilic monomers.

158. A cosmetic composition according to claim 144, wherein the hydrophilic monomers are chosen from cationic monomers, anionic monomers, and non-ionic monomers.

159. A cosmetic composition according to claim 145, wherein the non-hydrophilic monomers of the at least one block A are chosen from allylic monomers, acrylates, methacrylates, acrylamides, methacrylamides and vinyl monomers, and mixtures thereof, and optionally ethylenic monomers containing a lactam ring of formula (II).

160. A cosmetic composition according to claim 158, wherein the cationic monomers are chosen from 2-vinylpyridine; 4-vinylpyridine, dimethylaminoethyl (meth)acrylate; diethylaminoethyl (meth)acrylate; dimethylamino-propyl(meth)acrylamide; and salified or quaternized forms thereof.

161. A cosmetic composition according to claim 158, wherein the anionic monomers are chosen from acrylic acid, methacrylic acid, crotonic acid, maleic anhydride, itaconic acid, fumaric acid, maleic acid, styrenesulfonic acid, 2-acrylamido-2-methylpropanesulfonic acid, vinylbenzoic acid, vinylphosphonic acid and sulfopropyl (meth)acrylate, and the salts thereof.

162. A cosmetic composition according to claim 158, wherein the non-ionic monomers are chosen from:
ethylenic carboxybetaines and sulfobetaines,
hydroxyalkyl (meth)acrylates and hydroxyalkyl(meth)acrylamides, the alkyl group of which containing from 2 to 4 carbon atoms,
(meth)acrylates and (meth)acrylamides of (C₁-C₄)alkoxy(C₁₋₄)alkyl,
(meth)acrylates and (meth)acrylamides containing a group —(OC₂H₄)ₘ—OR″, in which m ranges from 5 to 150 and R″ is chosen from H and C₁ to C₄ alkyl, and
polysaccharide (meth)acrylates.

163. A cosmetic composition according to claim 162, wherein the ethylenic carboxybetaines or sulfobetaines are obtained by quaternization of ethylenically unsaturated monomers comprising an amine function with carboxylic acid salts containing a labile halogen or with cyclic sulfones.

164. A cosmetic composition according to claim 89, wherein said linear block ethylenic copolymer from chosen from diblock copolymers of AB type, triblock copolymers of ABA, BAB, ABC or ACB type, multiblock copolymers containing more than three blocks of (AB)ₙ, (ABA)ₙ, (BAB)ₙ or (ABC)ₙ type, with C other than A or B, and multiblock copolymers containing more than three different blocks, of ABCD type, wherein C is a block other than the blocks A and B.

165. A cosmetic composition according to claim 163, wherein the carboxylic acid salt containing a labile halogen is sodium chloroacetate.

166. A cosmetic composition according to claim 163, wherein the cyclic sulfone is propane sulfone.

167. A cosmetic composition according to claim 162, wherein the hydroxyalkyl (meth)acrylates and hydroxyalkyl(meth)acrylamides, the alkyl group of which contains from 2 to 4 carbon atoms, are chosen from hydroxyethyl (meth)acrylate and hydroxypropyl (meth)acrylate.

168. A cosmetic composition according to claim 162, wherein the (C₁-C₄)alkoxy(C₁₋₄)alkyl is chosen from methoxyethyl, ethoxyethyl and methoxypropyl.

169. A cosmetic composition according to claim 162, wherein the group —(OC₂H₄)ₘ—OR′″ is chosen from —POE-methoxy and —POE-OH.

170. A cosmetic composition according to claim 162, wherein the polysaccharide (meth)acrylate is sucrose acrylate.

171. A cosmetic composition according to claim 158, wherein the non-ionic monomers are chosen from vinyllactams.

172. A process for improving the hairstyle hold of a hair lacquer, without tack, comprising applying to hair a cosmetic composition comprising at least one linear block ethylenic copolymer comprising:
at least one block A derived from monomers comprising from 52 to 100% by weight of at least one ethylenic monomer containing a lactam ring, corresponding to formula (I) below: 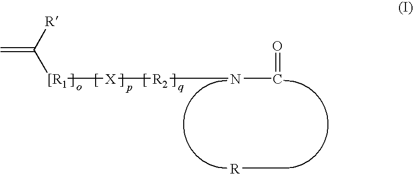
wherein:
R is a group —(CH₂)ₙ—, in which at least one of the carbon atoms is optionally replaced with a nitrogen atom or an oxygen atom, n is an integer chosen from 3 to 12, and at least one of the carbon atoms is optionally substituted with at least one C₁ to C₆ alkyl group;
R′ is chosen from H and a methyl group;
R₁ and R₂, which may be identical or different, are chosen from linear, branched and cyclic alkylene and aralkylene groups having 1 to 22 carbon atoms, in which at least one of the carbon atoms is optionally replaced with an oxygen or nitrogen atom;
X is chosen from —OCO—, —NHCO—, —COO— and —O—; and
o, p and q are chosen from, independently of each other, 0 and 1; wherein block A is present in an amount ranging from 1 to 99% by weight of the copolymer;
and at least one block B derived from monomers not comprising an ethylenic monomer containing a lactam ring of formula (I), or comprising a minor proportion thereof, the at least one block B having a glass transition temperature Tg ranging from −55° C. to +55° C.

173: A process according to claim 172, wherein o is 0, p is 1, q is 1, R₂ is —CH₂CH₂—; X is chosen from —COO— and —CONH—, and R is chosen from —(CH₂)₃—, —(CH₂)₅—, and —CH₂CH₂NH—.

174: A process according to claim 172, wherein the at least one ethylenic monomer contain in a lactam ring is a vinyllactam that corresponds to formula (II) below: 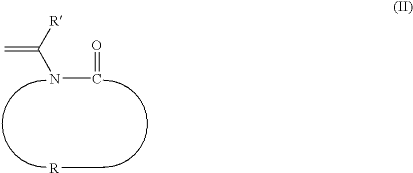
wherein:
R is a group —(CH₂)ₙ—, in which at least one of the carbon atoms is optionally replaced with a nitrogen atom or an oxygen atom, n is an integer chosen from 3 to 12, and at least one of the carbon atoms is optionally substituted with at least one C₁ to C₆ alkyl group; and
R′ is chosen from H and a methyl group.

175: A process according to claim 172, wherein R is —(CH₂)ₙ— in which n is an integer ranging form 3 to 5, or R is —CH₂—CH₂—NH—.

176: A process according to claim 174, wherein R is —(CH₂)ₙ— in which n is an integer from 3 to 5, or R is —CH₂—CH₂—NH—.

177: A cosmetic composition according to claim 89, wherein the at least one block B is derived from at least one ethylenic monomer chosen from: allylic monomers, acrylates, methacrylates, acrylamides, methacrylamides, and vinyl monomers.

178: A cosmetic composition according to claim 89, wherein the at least one block B monomer has a carbon chain length of less than or equal to 10 carbon atoms.

179: A cosmetic composition according to claim 89, wherein the at least one block B monomer has a carbon chain length of greater than or equal to 12 carbon atoms and the proportion of these monomers in the at least one block B is less than 50% by weight.

180: A cosmetic composition according to claim 89, wherein the at least one block B, with a glass transition temperature Tg ranging from −55° C. to +55° C., which is a homopolymer or a copolymer, is derived from at least one monomer, such that the homopolymer is prepared from these monomer have glass transition temperatures ranging from −200° C. to +250° C.

181: A cosmetic composition according to claim 153, wherein the first monomer is chosen from methyl methacrylate (MMA) and styrene.
